SEQUENCE LISTING (48 sequences)

SEQ ID NO: 1 - DNA, 384 bp, Artificial Sequence
  Description of Artificial Sequence Synthetic polynucleotide
caggtgcagctggtgcagtctggggctgaggtgaagaagcctgggtcctcggtgaaggtctcctgcaaggctcctggaggaaccttcagtagttatggtgtcaactgggtgcgacaggcccctggacaagggcttgagtggatgggagggatcgtccccatgtttggtgcagcagactacgcacagaaattccaacatagactcacaattaccgcggacgagtccacgaacacagcctacatggagctgagcagcctgagatctgaggacacggccgtctattactgtgcgagagcccgctgtcctattagcgactgcccccactctcattactattacttcatggacgtctggggcaaagggaccacggtcaccgtctcctca

SEQ ID NO: 2 - DNA, 384 bp, Artificial Sequence
  Description of Artificial Sequence Synthetic polynucleotide
caggtgcagctggtgcagtctggggctgaggtgaagaagcctgggtcctcggtgaaggtctcctgcaaggctcctggaggcaccttcaataactatggcatcaactgggtgcgacaggcccctggacaagggcttgagtggatgggagggatcgtccctatgatcggcgcaccaatgtacgcacagaaattcaagggcagagccacgattaccgcggacgcaaccacgagtacagtgtatatggagctgagcagcctgagatctgaggacacggccgtcttttattgtgcgagagcccgctgtcctggtatcgactgcccccattctgattattactacttcatggatgtctggggcaaggggaccacggtcaccgtctcctca

SEQ ID NO: 3 - DNA, 384 bp, Artificial Sequence
  Description of Artificial Sequence Synthetic polynucleotide
gaggtgcagctggtgcagtctggggctgaggtgaagaagcctgggtcctcggtgacggtctcctgcaaggctcctgaaggaaccttcactaattatggtgtcacctgggtgcgacaggcccctggacaagggcttgagtggatgggagggatcgtccctatgtttggtgcgccagactatgcaccgaaattccaggacagaatcacgattaccgcggacaagtccacgagcacagcctacatggaggtgagcagcctgagatccgaagacacggccgtctattactgtgcgagagtccgctgtggtgctttcgactgcccgcattctcattattacaattacatggacgtctggggcacagggaccacggtcaccgtctcctca

SEQ ID NO: 4 - DNA, 384 bp, Artificial Sequence
  Description of Artificial Sequence Synthetic polynucleotide
caggtgcagctggtgcagtctgggactgaggtgaagaagcctgggtcctcggtgacggtctcctgcaaggctcctggaggcaccttcaacacctatggtatcaactgggtgcgacaggcccctggacaaggccttgagtggatgggcgggatcgtccctatgtttggaatagcgacctacgcaccgaaattccaggacagactcagcattaccgcggacagatccacgagcacagccttcatgaaactgagcgccctgagatctgacgactcgggcgtcttttactgtgcgagagtccgctgtgatgctaacgactgtccgacgtccgatttctactactacatggacgtctggggcaaagggaccacggtcaccgtctcctca

SEQ ID NO: 5 - protein, 128 aa, Artificial Sequence
  Description of Artificial Sequence Synthetic polypeptide
QVQLVQSGAEVKKPGSSVKVSCKAPGGTFSSYGVNWVRQAPGQGLEWMGGIVPMFGAADYAQKFQHRLTITADESTNTAYMELSSLRSEDTAVYYCARARCPISDCPHSHYYYFMDVWGKGTTVTVSS

SEQ ID NO: 6 - protein, 128 aa, Artificial Sequence
  Description of Artificial Sequence Synthetic polypeptide
QVQLVQSGAEVKKPGSSVKVSCKAPGGTFNNYGINWVRQAPGQGLEWMGGIVPMIGAPMYAQKFKGRATITADATTSTVYMELSSLRSEDTAVFYCARARCPGIDCPHSDYYYFMDVWGKGTTVTVSS

SEQ ID NO: 7 - protein, 128 aa, Artificial Sequence
  Description of Artificial Sequence Synthetic polypeptide
EVQLVQSGAEVKKPGSSVTVSCKAPEGTFTNYGVTWVRQAPGQGLEWMGGIVPMFGAPDYAPKFQDRITITADKSTSTAYMEVSSLRSEDTAVYYCARVRCGAFDCPHSHYYNYMDVWGTGTTVTVSS

SEQ ID NO: 8 - protein, 128 aa, Artificial Sequence
  Description of Artificial Sequence Synthetic polypeptide
QVQLVQSGTEVKKPGSSVTVSCKAPGGTFNTYGINWVRQAPGQGLEWMGGIVPMFGIATYAPKFQDRLSITADRSTSTAFMKLSALRSDDSGVFYCARVRCDANDCPTSDFYYYMDVWGKGTTVTVSS

SEQ ID NO: 9 - DNA, 325 bp, Artificial Sequence
  Description of Artificial Sequence Synthetic polynucleotide
gaaattgtgttgacacagtctccagccaccctgtctttgtctccgggggaaagggccaccctctcctgcagggccagtcagagtattatcaggttcttagcctggtaccaacagcaacctggccaggctccaaggctcctcatctatgatgcatccaacagggccactggcatcccagccaggttcagtggcagtgggtctgggacagacttcactctcaccatcagcagcctagagcctgaagattttgcagtttattactgtcagctgcgtagcagtaatgatttcctcactttcggcggagggaccacggtggagatcaaac

SEQ ID NO: 10 - DNA, 319 bp, Artificial Sequence
  Description of Artificial Sequence Synthetic polynucleotide
gaaattgtgttgacacagtctccagccaccctgtctttgtctccgggggaaagtgccaccctctcctgcagggccagtcagagtattatcaggttcttagcctggtatcaacagagacctggccaggctcccaggctcctcatttatgatgcatccaacagggccgccggcatcccagccaggttcagtggcagtgggtctgggacagacttcactctcaccatcagcagcctagagcctgaagatttcgcagtttattactgtcagcaacatagcggcctcctcactttcggcggagggaccgccgtggagatcaaac

SEQ ID NO: 11 - DNA, 319 bp, Artificial Sequence
  Description of Artificial Sequence Synthetic polynucleotide
gaaattgtgttgacacagtctccagccaccctgtctttgtctccgggggaaagagccaccctctcctgcagggccagtcagagtgttatcaggttcttagcctggtaccaacagaaacctggccaggctcccaggctcctcatctatgatgcaaccgacagggaacctggcgtcccagccaggttcagtggcagtgggtctgggacagacttcagtctcaccatcagtagcctagagcctgaagattttgcggtctattactgtcagcagcgcagcagttccgtcactttcggcggagggaccacggtggagatcaaac

SEQ ID NO: 12 - DNA, 322 bp, Artificial Sequence
  Description of Artificial Sequence Synthetic polynucleotide
gaaattgtgttgacacagtctccagccaccctgtctttgtctcagggggagagagccgccctctcctgcagggccagtcagagtgtaatcaggttcttagcctggtaccaacagaaacctggccaggctcccaggctcctcatatatgatgcatccaacagggccactggcaccccagccaggttcagtggcagtgggtctgggacagacttcactctcaccatcagcagcctagagcctgaagattttgcggtttattactgtcagcagcgtagcggcggcctcgtcactttcggcggagggaccacggtgcaggtcaaac

SEQ ID NO: 13 - protein, 108 aa, Artificial Sequence
  Description of Artificial Sequence Synthetic polypeptide
EIVLTQSPATLSLSPGERATLSCRASQSIIRFLAWYQQQPGQAPRLLIYDASNRATGIPARFSGSGSGTDFTLTISSLEPEDFAVYYCQLRSSNDFLTFGGGTTVEIK

SEQ ID NO: 14 - protein, 106 aa, Artificial Sequence
  Description of Artificial Sequence Synthetic polypeptide
EIVLTQSPATLSLSPGESATLSCRASQSIIRFLAWYQQRPGQAPRLLIYDASNRAAGIPARFSGSGSGTDFTLTISSLEPEDFAVYYCQQHSGLLTFGGGTAVEIK

SEQ ID NO: 15 - protein, 106 aa, Artificial Sequence
  Description of Artificial Sequence Synthetic polypeptide
EIVLTQSPATLSLSPGERATLSCRASQSVIRFLAWYQQKPGQAPRLLIYDATDREPGVPARFSGSGSGTDFSLTISSLEPEDFAVYYCQQRSSSVTFGGGTTVEIK

SEQ ID NO: 16 - protein, 107 aa, Artificial Sequence
  Description of Artificial Sequence Synthetic polypeptide
EIVLTQSPATLSLSQGERAALSCRASQSVIRFLAWYQQKPGQAPRLLIYDASNRATGTPARFSGSGSGTDFTLTISSLEPEDFAVYYCQQRSGGLVTFGGGTTVQVK

SEQ ID NO: 17 - DNA, 382 bp, Artificial Sequence
  Description of Artificial Sequence Synthetic polynucleotide
caggtgcagctgcaggagtcgggcccaggactggtgaagccttcggagaccctgtccctcgcctgcagtgtctctggtgactccatcagcagcagtagatactactgggactggatccgccaggcccccgggaaggggctggagtggattgggggtttctactattatgagtctagtgagagagttttctacaatccgtccctcaagagtcgacccaccgtatccgttgacacctccaagaaccagttctccctgagactgacttctgttaccgccgcagacacggctatttattactgtgcgagacgccagtttgattccgggacccctgagggctggttcgacccctggggccagggaaccctggtcaccgtctcctcag

SEQ ID NO: 18 - protein, 127 aa, Artificial Sequence
  Description of Artificial Sequence Synthetic polypeptide
QVQLQESGPGLVKPSETLSLACSVSGDSISSSRYYWDWIRQAPGKGLEWIGGFYYYESSERVFYNPSLKSRPTVSVDTSKNQFSLRLTSVTAADTAIYYCARRQFDSGTPEGWFDPWGQGTLVTVSS

SEQ ID NO: 19 - DNA, 337 bp, Artificial Sequence
  Description of Artificial Sequence Synthetic polynucleotide
cagtctgtgctgacgcagccgccctcagtgtctggggccccagggcagagggtcaccatctcctgcactgggagcggctccaacatcggggcagattatgatgtgcactggtaccagcaggttccaggaacagcccccaaactcctcatctatggtgacaccaatcggccctcaggggtccctgaccgattttccggctccaggtctggcacctcagcctccctggtcatcactggactccaggctgaggatgaggctgattattactgccagtcctatgacaacggcctgagtggtttttgggtgttcggcggagggaccactctgaccgtcctgc

SEQ ID NO: 20 - protein, 112 aa, Artificial Sequence
  Description of Artificial Sequence Synthetic polypeptide
QSVLTQPPSVSGAPGQRVTISCTGSGSNIGADYDVHWYQQVPGTAPKLLIYGDTNRPSGVPDRFSGSRSGTSASLVITGLQAEDEADYYCQSYDNGLSGFWVFGGGTTLTVL

SEQ ID NO: 21 - DNA, 390 bp, Artificial Sequence
  Description of Artificial Sequence Synthetic polynucleotide
gaggtgcagctggtgcagtctggggctgaggtgaagaagcctgggtcctcggtgaaggtctcctgtaaggcgtctggagacgccatcagagaccatgctattagttgggtgcgacaggcccctggacaaggccttgagtggatgggagggatcgtctcaggttttggtgcagcaaactacgcacagaagttccagggcagagtcaccatcaccgcggacgactccacgaacacagtctacatggaggtgccccgcctgagatctgacgacacggccgtatattattgtgcgaaatcggtctccgcggacagccacgacaaacccacggttgacttctacttctactacatggacgtctggggcacggggaccaccgtcaccgtctcctca

SEQ ID NO: 22 - protein, 130 aa, Artificial Sequence
  Description of Artificial Sequence Synthetic polypeptide
EVQLVQSGAEVKKPGSSVKVSCKASGDAIRDHAISWVRQAPGQGLEWMGGIVSGFGAANYAQKFQGRVTITADDSTNTVYMEVPRLRSDDTAVYYCAKSVSADSHDKPTVDFYFYYMDVWGTGTTVTVSS

SEQ ID NO: 23 - DNA, 346 bp, Artificial Sequence
  Description of Artificial Sequence Synthetic polynucleotide
gacatcgtgatgacccagtctccagactccctggctgtgtctctgggcgagagggccaccatcaactgcaagtccagccagagtattctattcagcgccctcaatttgaattacttagcttggtaccagcagaaaccaggtcagcctcctaggttgctcatctactgggcatctacccgggaatccggagtccctgaccgattcagtggcagcgggtctgggacagacttcactctcaccatcagcagcctgcaggctgaagatgtggcagtttattactgtcagcaatattatgatagtcctccgggactcactttcggcggagggaccagggtggaaatcaaac

SEQ ID NO: 24 - protein, 115 aa, Artificial Sequence
  Description of Artificial Sequence Synthetic polypeptide
DIVMTQSPDSLAVSLGERATINCKSSQSILFSALNLNYLAWYQQKPGQPPRLLIYWASTRESGVPDRFSGSGSGTDFTLTISSLQAEDVAVYYCQQYYDSPPGLTFGGGTRVEIK

SEQ ID NO: 25 - RNA, 1386 nt, Artificial Sequence
  Description of Artificial Sequence Synthetic polynucleotide
caggugcagcuggugcaguccggcgccgaggugaagaagcccggcuccuccgugaagguguccugcaaggcccccggcggcaccuucaacaacuacggcaucaacugggugcgccaggcccccggccagggccuggaguggaugggcggcaucgugcccaugaucggcgcccccauguacgcccagaaguucaagggccgcgccaccaucaccgccgacgccaccaccuccaccguguacauggagcuguccucccugcgcuccgaggacaccgccguguucuacugcgcccgcgcccgcugccccggcaucgacugcccccacuccgacuacuacuacuucauggacguguggggcaagggcaccaccgugaccguguccuccggcgaccccuccgccuccaccaagggccccuccguguucccccuggcccccuccuccaaguccaccuccggcggcaccgccgcccugggcugccuggugaaggacuacuuccccgagcccgugaccguguccuggaacuccggcgcccugaccuccggcgugcacaccuuccccgccgugcugcaguccuccggccuguacucccuguccuccguggugaccgugcccuccuccucccugggcacccagaccuacaucugcaacgugaaccacaagcccuccaacaccaagguggacaagaagguggagcccaaguccugcgacaagacccacaccugcccccccugccccgcccccgagcugcugggcggccccuccguguuccuguucccccccaagcccaaggacacccugaugaucucccgcacccccgaggugaccugcguggugguggacgugucccacgaggaccccgaggugaaguucaacugguacguggacggcguggaggugcacaacgccaagaccaagccccgcgaggagcaguacaacuccaccuaccgcgugguguccgugcugaccgugcugcaccaggacuggcugaacggcaaggaguacaagugcaagguguccaacaaggcccugcccgcccccaucgagaagaccaucuccaaggccaagggccagccccgcgagccccagguguacacccugccccccucccgcgacgagcugaccaagaaccaggugucccugaccugccuggugaagggcuucuaccccuccgacaucgccguggagugggaguccaacggccagcccgagaacaacuacaagaccaccccccccgugcuggacuccgacggcuccuucuuccuguacuccaagcugaccguggacaagucccgcuggcagcagggcaacguguucuccugcuccgugaugcacgaggcccugcacaaccacuacacccagaagucccugucccugucccccggcaag

SEQ ID NO: 26 - RNA, 652 nt, Artificial Sequence
  Description of Artificial Sequence Synthetic polynucleotide
gagaucgugcugacccagucccccgccacccugucccugucccccggcgaguccgccacccuguccugccgcgccucccaguccaucauccgcuuccuggccugguaccagcagcgccccggccaggccccccgccugcugaucuacgacgccuccaaccgcgccgccggcauccccgcccgcuucuccggcuccggcuccggcaccgacuucacccugaccaucuccucccuggagcccgaggacuucgccguguacuacugccagcagcacuccggccugcugaccuucggcggcggcaccgccguggagaucaaagggcgaccccucccgcaccguggccgcccccuccguguucaucuucccccccuccgacgagcagcugaaguccggcaccgccuccguggugugccugcugaacaacuucuacccccgcgaggccaaggugcaguggaagguggacaacgcccugcaguccggcaacucccaggaguccgugaccgagcaggacuccaaggacuccaccuacucccuguccuccacccugacccuguccaaggccgacuacgagaagcacaagguguacgccugcgaggugacccaccagggccuguccucccccgugaccaaguccuucaaccgcggcgagugc

SEQ ID NO: 27 - RNA, 1386 nt, Artificial Sequence
  Description of Artificial Sequence Synthetic polynucleotide
caggugcagcuggugcaguccggcgccgaggugaagaagcccggcuccuccgugaagguguccugcaaggcccccggcggcaccuucuccuccuacggcgugaacugggugcgccaggcccccggccagggccuggaguggaugggcggcaucgugcccauguucggcgccgccgacuacgcccagaaguuccagcaccgccugaccaucaccgccgacgaguccaccaacaccgccuacauggagcuguccucccugcgcuccgaggacaccgccguguacuacugcgcccgcgcccgcugccccaucuccgacugcccccacucccacuacuacuacuucauggacguguggggcaagggcaccaccgugaccguguccuccggcgaccccuccgccuccaccaagggccccuccguguucccccuggcccccuccuccaaguccaccuccggcggcaccgccgcccugggcugccuggugaaggacuacuuccccgagcccgugaccguguccuggaacuccggcgcccugaccuccggcgugcacaccuuccccgccgugcugcaguccuccggccuguacucccuguccuccguggugaccgugcccuccuccucccugggcacccagaccuacaucugcaacgugaaccacaagcccuccaacaccaagguggacaagaagguggagcccaaguccugcgacaagacccacaccugcccccccugccccgcccccgagcugcugggcggccccuccguguuccuguucccccccaagcccaaggacacccugaugaucucccgcacccccgaggugaccugcguggugguggacgugucccacgaggaccccgaggugaaguucaacugguacguggacggcguggaggugcacaacgccaagaccaagccccgcgaggagcaguacaacuccaccuaccgcgugguguccgugcugaccgugcugcaccaggacuggcugaacggcaaggaguacaagugcaagguguccaacaaggcccugcccgcccccaucgagaagaccaucuccaaggccaagggccagccccgcgagccccagguguacacccugccccccucccgcgacgagcugaccaagaaccaggugucccugaccugccuggugaagggcuucuaccccuccgacaucgccguggagugggaguccaacggccagcccgagaacaacuacaagaccaccccccccgugcuggacuccgacggcuccuucuuccuguacuccaagcugaccguggacaagucccgcuggcagcagggcaacguguucuccugcuccgugaugcacgaggcccugcacaaccacuacacccagaagucccugucccugucccccggcaag

SEQ ID NO: 28 - RNA, 658 nt, Artificial Sequence
  Description of Artificial Sequence Synthetic polynucleotide
gagaucgugcugacccagucccccgccacccugucccugucccccggcgagcgcgccacccuguccugccgcgccucccaguccaucauccgcuuccuggccugguaccagcagcagcccggccaggccccccgccugcugaucuacgacgccuccaaccgcgccaccggcauccccgcccgcuucuccggcuccggcuccggcaccgacuucacccugaccaucuccucccuggagcccgaggacuucgccguguacuacugccagcugcgcuccuccaacgacuuccugaccuucggcggcggcaccaccguggagaucaaagggcgaccccucccgcaccguggccgcccccuccguguucaucuucccccccuccgacgagcagcugaaguccggcaccgccuccguggugugccugcugaacaacuucuacccccgcgaggccaaggugcaguggaagguggacaacgcccugcaguccggcaacucccaggaguccgugaccgagcaggacuccaaggacuccaccuacucccuguccuccacccugacccuguccaaggccgacuacgagaagcacaagguguacgccugcgaggugacccaccagggccuguccucccccgugaccaaguccuucaaccgcggcgagugc

SEQ ID NO: 29 - RNA, 1386 nt, Artificial Sequence
  Description of Artificial Sequence Synthetic polynucleotide
caggugcagcuggugcaguccggcaccgaggugaagaagcccggcuccuccgugaccguguccugcaaggcccccggcggcaccuucaacaccuacggcaucaacugggugcgccaggcccccggccagggccuggaguggaugggcggcaucgugcccauguucggcaucgccaccuacgcccccaaguuccaggaccgccuguccaucaccgccgaccgcuccaccuccaccgccuucaugaagcuguccgcccugcgcuccgacgacuccggcguguucuacugcgcccgcgugcgcugcgacgccaacgacugccccaccuccgacuucuacuacuacauggacguguggggcaagggcaccaccgugaccguguccuccggcgaccccuccgccuccaccaagggccccuccguguucccccuggcccccuccuccaaguccaccuccggcggcaccgccgcccugggcugccuggugaaggacuacuuccccgagcccgugaccguguccuggaacuccggcgcccugaccuccggcgugcacaccuuccccgccgugcugcaguccuccggccuguacucccuguccuccguggugaccgugcccuccuccucccugggcacccagaccuacaucugcaacgugaaccacaagcccuccaacaccaagguggacaagaagguggagcccaaguccugcgacaagacccacaccugcccccccugccccgcccccgagcugcugggcggccccuccguguuccuguucccccccaagcccaaggacacccugaugaucucccgcacccccgaggugaccugcguggugguggacgugucccacgaggaccccgaggugaaguucaacugguacguggacggcguggaggugcacaacgccaagaccaagccccgcgaggagcaguacaacuccaccuaccgcgugguguccgugcugaccgugcugcaccaggacuggcugaacggcaaggaguacaagugcaagguguccaacaaggcccugcccgcccccaucgagaagaccaucuccaaggccaagggccagccccgcgagccccagguguacacccugccccccucccgcgacgagcugaccaagaaccaggugucccugaccugccuggugaagggcuucuaccccuccgacaucgccguggagugggaguccaacggccagcccgagaacaacuacaagaccaccccccccgugcuggacuccgacggcuccuucuuccuguacuccaagcugaccguggacaagucccgcuggcagcagggcaacguguucuccugcuccgugaugcacgaggcccugcacaaccacuacacccagaagucccugucccugucccccggcaag

SEQ ID NO: 30 - RNA, 655 nt, Artificial Sequence
  Description of Artificial Sequence Synthetic polynucleotide
gagaucgugcugacccagucccccgccacccugucccugucccagggcgagcgcgccgcccuguccugccgcgccucccaguccgugauccgcuuccuggccugguaccagcagaagcccggccaggccccccgccugcugaucuacgacgccuccaaccgcgccaccggcacccccgcccgcuucuccggcuccggcuccggcaccgacuucacccugaccaucuccucccuggagcccgaggacuucgccguguacuacugccagcagcgguccggcggccuggugaccuucggcggcggcaccaccgugcaggugaaagggcgaccccucccgcaccguggccgcccccuccguguucaucuucccccccuccgacgagcagcugaaguccggcaccgccuccguggugugccugcugaacaacuucuacccccgcgaggccaaggugcaguggaagguggacaacgcccugcaguccggcaacucccaggaguccgugaccgagcaggacuccaaggacuccaccuacucccuguccuccacccugacccuguccaaggccgacuacgagaagcacaagguguacgccugcgaggugacccaccagggccuguccucccccgugaccaaguccuucaaccgcggcgagugc

SEQ ID NO: 31 - RNA, 1386 nt, Artificial Sequence
  Description of Artificial Sequence Synthetic polynucleotide
gaggugcagcuggugcaguccggcgccgaggugaagaagcccggcuccuccgugaccguguccugcaaggcccccgagggcaccuucaccaacuacggcgugaccugggugcgccaggcccccggccagggccuggaguggaugggcggcaucgugcccauguucggcgcccccgacuacgcccccaaguuccaggaccgcaucaccaucaccgccgacaaguccaccuccaccgccuacauggagguguccucccugcgcuccgaggacaccgccguguacuacugcgcccgcgugcgcugcggcgccuucgacugcccccacucccacuacuacaacuacauggacguguggggcaccggcaccaccgugaccguguccuccggcgaccccuccgccuccaccaagggccccuccguguucccccuggcccccuccuccaaguccaccuccggcggcaccgccgcccugggcugccuggugaaggacuacuuccccgagcccgugaccguguccuggaacuccggcgcccugaccuccggcgugcacaccuuccccgccgugcugcaguccuccggccuguacucccuguccuccguggugaccgugcccuccuccucccugggcacccagaccuacaucugcaacgugaaccacaagcccuccaacaccaagguggacaagaagguggagcccaaguccugcgacaagacccacaccugcccccccugccccgcccccgagcugcugggcggccccuccguguuccuguucccccccaagcccaaggacacccugaugaucucccgcacccccgaggugaccugcguggugguggacgugucccacgaggaccccgaggugaaguucaacugguacguggacggcguggaggugcacaacgccaagaccaagccccgcgaggagcaguacaacuccaccuaccgcgugguguccgugcugaccgugcugcaccaggacuggcugaacggcaaggaguacaagugcaagguguccaacaaggcccugcccgcccccaucgagaagaccaucuccaaggccaagggccagccccgcgagccccagguguacacccugccccccucccgcgacgagcugaccaagaaccaggugucccugaccugccuggugaagggcuucuaccccuccgacaucgccguggagugggaguccaacggccagcccgagaacaacuacaagaccaccccccccgugcuggacuccgacggcuccuucuuccuguacuccaagcugaccguggacaagucccgcuggcagcagggcaacguguucuccugcuccgugaugcacgaggcccugcacaaccacuacacccagaagucccugucccugucccccggcaag

SEQ ID NO: 32 - RNA, 652 nt, Artificial Sequence
  Description of Artificial Sequence Synthetic polynucleotide
gagaucgugcugacccagucccccgccacccugucccugucccccggcgagcgcgccacccuguccugccgcgccucccaguccgugauccgcuuccuggccugguaccagcagaagcccggccaggccccccgccugcugaucuacgacgccaccgaccgcgagcccggcgugcccgcccgcuucuccggcuccggcuccggcaccgacuucucccugaccaucuccucccuggagcccgaggacuucgccguguacuacugccagcagcgguccuccuccgugaccuucggcggcggcaccaccguggagaucaaagggcgaccccucccgcaccguggccgcccccuccguguucaucuucccccccuccgacgagcagcugaaguccggcaccgccuccguggugugccugcugaacaacuucuacccccgcgaggccaaggugcaguggaagguggacaacgcccugcaguccggcaacucccaggaguccgugaccgagcaggacuccaaggacuccaccuacucccuguccuccacccugacccuguccaaggccgacuacgagaagcacaagguguacgccugcgaggugacccaccagggccuguccucccccgugaccaaguccuucaaccgcggcgagugc

SEQ ID NO: 33 - RNA, 1383 nt, Artificial Sequence
  Description of Artificial Sequence Synthetic polynucleotide
caggugcagcugcaggaguccggccccggccuggugaagcccuccgagacccugucccuggccugcuccguguccggcgacuccaucuccuccucccgcuacuacugggacuggauccgccaggcccccggcaagggccuggaguggaucggcggcuucuacuacuacgaguccuccgagcgcguguucuacaaccccucccugaagucccgccccaccguguccguggacaccuccaagaaccaguucucccugcgccugaccuccgugaccgccgccgacaccgccaucuacuacugcgcccgccgccaguucgacuccggcacccccgagggcugguucgaccccuggggccagggcacccuggugaccguguccuccggcgaccccuccgccuccaccaagggccccuccguguucccccuggcccccuccuccaaguccaccuccggcggcaccgccgcccugggcugccuggugaaggacuacuuccccgagcccgugaccguguccuggaacuccggcgcccugaccuccggcgugcacaccuuccccgccgugcugcaguccuccggccuguacucccuguccuccguggugaccgugcccuccuccucccugggcacccagaccuacaucugcaacgugaaccacaagcccuccaacaccaagguggacaagaagguggagcccaaguccugcgacaagacccacaccugcccccccugccccgcccccgagcugcugggcggccccuccguguuccuguucccccccaagcccaaggacacccugaugaucucccgcacccccgaggugaccugcguggugguggacgugucccacgaggaccccgaggugaaguucaacugguacguggacggcguggaggugcacaacgccaagaccaagccccgcgaggagcaguacaacuccaccuaccgcgugguguccgugcugaccgugcugcaccaggacuggcugaacggcaaggaguacaagugcaagguguccaacaaggcccugcccgcccccaucgagaagaccaucuccaaggccaagggccagccccgcgagccccagguguacacccugccccccucccgcgacgagcugaccaagaaccaggugucccugaccugccuggugaagggcuucuaccccuccgacaucgccguggagugggaguccaacggccagcccgagaacaacuacaagaccaccccccccgugcuggacuccgacggcuccuucuuccuguacuccaagcugaccguggacaagucccgcuggcagcagggcaacguguucuccugcuccgugaugcacgaggcccugcacaaccacuacacccagaagucccugucccugucccccggcaag

SEQ ID NO: 34 - RNA, 651 nt, Artificial Sequence
  Description of Artificial Sequence Synthetic polynucleotide
caguccgugcugacccagccccccuccguguccggcgcccccggccagcgcgugaccaucuccugcaccggcuccggcuccaacaucggcgccgacuacgacgugcacugguaccagcaggugcccggcaccgcccccaagcugcugaucuacggcgacaccaaccgccccuccggcgugcccgaccgcuucuccggcucccgcuccggcaccuccgccucccuggugaucaccggccugcaggccgaggacgaggccgacuacuacugccaguccuacgacaacggccuguccggcuucuggguguucggcggcggcaccacccugaccgugcugggccagcccaaggccgcccccucggucacccuguucccgcccuccuccgaggagcuccaagccaacaaggccacacuggugugccucauaagcgacuucuacccgggagccgugacaguggccuggaaggcagacagcagccccgucaaggcgggaguggagaccaccacacccuccaaacaaagcaacaacaaguacgcggccagcagcuaccugagccugacgcccgagcaguggaagucccacagaagcuacagcugccaggucacgcacgaagggagcaccguggagaagacaguggcccccacagaaugc

SEQ ID NO: 35 - RNA, 1392 nt, Artificial Sequence
  Description of Artificial Sequence Synthetic polynucleotide
gaggugcagcuggugcaguccggcgccgaggugaagaagcccggcuccuccgugaagguguccugcaaggccuccggcgacgccauccgcgaccacgccaucuccugggugcgccaggcccccggccagggccuggaguggaugggcggcaucguguccggcuucggcgccgccaacuacgcccagaaguuccagggccgcgugaccaucaccgccgacgacuccaccaacaccguguacauggaggugccccgccugcgcuccgacgacaccgccguguacuacugcgccaaguccguguccgccgacucccacgacaagcccaccguggacuucuacuucuacuacauggacguguggggcaccggcaccaccgugaccguguccuccggcgaccccuccgccuccaccaagggccccuccguguucccccuggcccccuccuccaaguccaccuccggcggcaccgccgcccugggcugccuggugaaggacuacuuccccgagcccgugaccguguccuggaacuccggcgcccugaccuccggcgugcacaccuuccccgccgugcugcaguccuccggccuguacucccuguccuccguggugaccgugcccuccuccucccugggcacccagaccuacaucugcaacgugaaccacaagcccuccaacaccaagguggacaagaagguggagcccaaguccugcgacaagacccacaccugcccccccugccccgcccccgagcugcugggcggccccuccguguuccuguucccccccaagcccaaggacacccugaugaucucccgcacccccgaggugaccugcguggugguggacgugucccacgaggaccccgaggugaaguucaacugguacguggacggcguggaggugcacaacgccaagaccaagccccgcgaggagcaguacaacuccaccuaccgcgugguguccgugcugaccgugcugcaccaggacuggcugaacggcaaggaguacaagugcaagguguccaacaaggcccugcccgcccccaucgagaagaccaucuccaaggccaagggccagccccgcgagccccagguguacacccugccccccucccgcgacgagcugaccaagaaccaggugucccugaccugccuggugaagggcuucuaccccuccgacaucgccguggagugggaguccaacggccagcccgagaacaacuacaagaccaccccccccgugcuggacuccgacggcuccuucuuccuguacuccaagcugaccguggacaagucccgcuggcagcagggcaacguguucuccugcuccgugaugcacgaggcccugcacaaccacuacacccagaagucccugucccugucccccggcaag

SEQ ID NO: 36 - RNA, 678 nt, Artificial Sequence
  Description of Artificial Sequence Synthetic polynucleotide
gacaucgugaugacccagucccccgacucccuggccgugucccugggcgagcgcgccaccaucaacugcaaguccucccaguccauccuguucuccgcccugaaccugaacuaccuggccugguaccagcagaagcccggccagcccccccgccugcugaucuacugggccuccacccgcgaguccggcgugcccgaccgcuucuccggcuccggcuccggcaccgacuucacccugaccaucuccucccugcaggccgaggacguggccguguacuacugccagcaguacuacgacuccccccccggccugaccuucggcggcggcacccgcguggagaucaagggcgaccccucccgcaccguggccgcccccuccguguucaucuucccccccuccgacgagcagcugaaguccggcaccgccuccguggugugccugcugaacaacuucuacccccgcgaggccaaggugcaguggaagguggacaacgcccugcaguccggcaacucccaggaguccgugaccgagcaggacuccaaggacuccaccuacucccuguccuccacccugacccuguccaaggccgacuacgagaagcacaagguguacgccugcgaggugacccaccagggccuguccucccccgugaccaaguccuucaaccgcggcgagugc

SEQ ID NO: 37 - DNA, 1386 bp, Artificial Sequence
  Description of Artificial Sequence Synthetic polynucleotide
caggtgcagctggtgcagtccggcgccgaggtgaagaagcccggctcctccgtgaaggtgtcctgcaaggcccccggcggcaccttcaacaactacggcatcaactgggtgcgccaggcccccggccagggcctggagtggatgggcggcatcgtgcccatgatcggcgcccccatgtacgcccagaagttcaagggccgcgccaccatcaccgccgacgccaccacctccaccgtgtacatggagctgtcctccctgcgctccgaggacaccgccgtgttctactgcgcccgcgcccgctgccccggcatcgactgcccccactccgactactactacttcatggacgtgtggggcaagggcaccaccgtgaccgtgtcctccggcgacccctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccgcacccccgaggtgacctgcgtggtggtggacgtgtcccacgaggaccccgaggtgaagttcaactggtacgtggacggcgtggaggtgcacaacgccaagaccaagccccgcgaggagcagtacaactccacctaccgcgtggtgtccgtgctgaccgtgctgcaccaggactggctgaacggcaaggagtacaagtgcaaggtgtccaacaaggccctgcccgcccccatcgagaagaccatctccaaggccaagggccagccccgcgagccccaggtgtacaccctgcccccctcccgcgacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccgctggcagcagggcaacgtgttctcctgctccgtgatgcacgaggccctgcacaaccactacacccagaagtccctgtccctgtcccccggcaag

SEQ ID NO: 38 - DNA, 652 bp, Artificial Sequence
  Description of Artificial Sequence Synthetic polynucleotide
gagatcgtgctgacccagtcccccgccaccctgtccctgtcccccggcgagtccgccaccctgtcctgccgcgcctcccagtccatcatccgcttcctggcctggtaccagcagcgccccggccaggccccccgcctgctgatctacgacgcctccaaccgcgccgccggcatccccgcccgcttctccggctccggctccggcaccgacttcaccctgaccatctcctccctggagcccgaggacttcgccgtgtactactgccagcagcactccggcctgctgaccttcggcggcggcaccgccgtggagatcaaagggcgacccctcccgcaccgtggccgccccctccgtgttcatcttccccccctccgacgagcagctgaagtccggcaccgcctccgtggtgtgcctgctgaacaacttctacccccgcgaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccgcggcgagtgc

SEQ ID NO: 39 - DNA, 1386 bp, Artificial Sequence
  Description of Artificial Sequence Synthetic polynucleotide
caggtgcagctggtgcagtccggcgccgaggtgaagaagcccggctcctccgtgaaggtgtcctgcaaggcccccggcggcaccttctcctcctacggcgtgaactgggtgcgccaggcccccggccagggcctggagtggatgggcggcatcgtgcccatgttcggcgccgccgactacgcccagaagttccagcaccgcctgaccatcaccgccgacgagtccaccaacaccgcctacatggagctgtcctccctgcgctccgaggacaccgccgtgtactactgcgcccgcgcccgctgccccatctccgactgcccccactcccactactactacttcatggacgtgtggggcaagggcaccaccgtgaccgtgtcctccggcgacccctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccgcacccccgaggtgacctgcgtggtggtggacgtgtcccacgaggaccccgaggtgaagttcaactggtacgtggacggcgtggaggtgcacaacgccaagaccaagccccgcgaggagcagtacaactccacctaccgcgtggtgtccgtgctgaccgtgctgcaccaggactggctgaacggcaaggagtacaagtgcaaggtgtccaacaaggccctgcccgcccccatcgagaagaccatctccaaggccaagggccagccccgcgagccccaggtgtacaccctgcccccctcccgcgacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccgctggcagcagggcaacgtgttctcctgctccgtgatgcacgaggccctgcacaaccactacacccagaagtccctgtccctgtcccccggcaag

SEQ ID NO: 40 - DNA, 658 bp, Artificial Sequence
  Description of Artificial Sequence Synthetic polynucleotide
gagatcgtgctgacccagtcccccgccaccctgtccctgtcccccggcgagcgcgccaccctgtcctgccgcgcctcccagtccatcatccgcttcctggcctggtaccagcagcagcccggccaggccccccgcctgctgatctacgacgcctccaaccgcgccaccggcatccccgcccgcttctccggctccggctccggcaccgacttcaccctgaccatctcctccctggagcccgaggacttcgccgtgtactactgccagctgcgctcctccaacgacttcctgaccttcggcggcggcaccaccgtggagatcaaagggcgacccctcccgcaccgtggccgccccctccgtgttcatcttccccccctccgacgagcagctgaagtccggcaccgcctccgtggtgtgcctgctgaacaacttctacccccgcgaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccgcggcgagtgc

SEQ ID NO: 41 - DNA, 1386 bp, Artificial Sequence
  Description of Artificial Sequence Synthetic polynucleotide
caggtgcagctggtgcagtccggcaccgaggtgaagaagcccggctcctccgtgaccgtgtcctgcaaggcccccggcggcaccttcaacacctacggcatcaactgggtgcgccaggcccccggccagggcctggagtggatgggcggcatcgtgcccatgttcggcatcgccacctacgcccccaagttccaggaccgcctgtccatcaccgccgaccgctccacctccaccgccttcatgaagctgtccgccctgcgctccgacgactccggcgtgttctactgcgcccgcgtgcgctgcgacgccaacgactgccccacctccgacttctactactacatggacgtgtggggcaagggcaccaccgtgaccgtgtcctccggcgacccctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccgcacccccgaggtgacctgcgtggtggtggacgtgtcccacgaggaccccgaggtgaagttcaactggtacgtggacggcgtggaggtgcacaacgccaagaccaagccccgcgaggagcagtacaactccacctaccgcgtggtgtccgtgctgaccgtgctgcaccaggactggctgaacggcaaggagtacaagtgcaaggtgtccaacaaggccctgcccgcccccatcgagaagaccatctccaaggccaagggccagccccgcgagccccaggtgtacaccctgcccccctcccgcgacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccgctggcagcagggcaacgtgttctcctgctccgtgatgcacgaggccctgcacaaccactacacccagaagtccctgtccctgtcccccggcaag

SEQ ID NO: 42 - DNA, 655 bp, Artificial Sequence
  Description of Artificial Sequence Synthetic polynucleotide
gagatcgtgctgacccagtcccccgccaccctgtccctgtcccagggcgagcgcgccgccctgtcctgccgcgcctcccagtccgtgatccgcttcctggcctggtaccagcagaagcccggccaggccccccgcctgctgatctacgacgcctccaaccgcgccaccggcacccccgcccgcttctccggctccggctccggcaccgacttcaccctgaccatctcctccctggagcccgaggacttcgccgtgtactactgccagcagcggtccggcggcctggtgaccttcggcggcggcaccaccgtgcaggtgaaagggcgacccctcccgcaccgtggccgccccctccgtgttcatcttccccccctccgacgagcagctgaagtccggcaccgcctccgtggtgtgcctgctgaacaacttctacccccgcgaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccgcggcgagtgc

SEQ ID NO: 43 - DNA, 1386 bp, Artificial Sequence
  Description of Artificial Sequence Synthetic polynucleotide
gaggtgcagctggtgcagtccggcgccgaggtgaagaagcccggctcctccgtgaccgtgtcctgcaaggcccccgagggcaccttcaccaactacggcgtgacctgggtgcgccaggcccccggccagggcctggagtggatgggcggcatcgtgcccatgttcggcgcccccgactacgcccccaagttccaggaccgcatcaccatcaccgccgacaagtccacctccaccgcctacatggaggtgtcctccctgcgctccgaggacaccgccgtgtactactgcgcccgcgtgcgctgcggcgccttcgactgcccccactcccactactacaactacatggacgtgtggggcaccggcaccaccgtgaccgtgtcctccggcgacccctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccgcacccccgaggtgacctgcgtggtggtggacgtgtcccacgaggaccccgaggtgaagttcaactggtacgtggacggcgtggaggtgcacaacgccaagaccaagccccgcgaggagcagtacaactccacctaccgcgtggtgtccgtgctgaccgtgctgcaccaggactggctgaacggcaaggagtacaagtgcaaggtgtccaacaaggccctgcccgcccccatcgagaagaccatctccaaggccaagggccagccccgcgagccccaggtgtacaccctgcccccctcccgcgacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccgctggcagcagggcaacgtgttctcctgctccgtgatgcacgaggccctgcacaaccactacacccagaagtccctgtccctgtcccccggcaag

SEQ ID NO: 44 - DNA, 652 bp, Artificial Sequence
  Description of Artificial Sequence Synthetic polynucleotide
gagatcgtgctgacccagtcccccgccaccctgtccctgtcccccggcgagcgcgccaccctgtcctgccgcgcctcccagtccgtgatccgcttcctggcctggtaccagcagaagcccggccaggccccccgcctgctgatctacgacgccaccgaccgcgagcccggcgtgcccgcccgcttctccggctccggctccggcaccgacttctccctgaccatctcctccctggagcccgaggacttcgccgtgtactactgccagcagcggtcctcctccgtgaccttcggcggcggcaccaccgtggagatcaaagggcgacccctcccgcaccgtggccgccccctccgtgttcatcttccccccctccgacgagcagctgaagtccggcaccgcctccgtggtgtgcctgctgaacaacttctacccccgcgaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccgcggcgagtgc

SEQ ID NO: 45 - DNA, 1383 bp, Artificial Sequence
  Description of Artificial Sequence Synthetic polynucleotide
caggtgcagctgcaggagtccggccccggcctggtgaagccctccgagaccctgtccctggcctgctccgtgtccggcgactccatctcctcctcccgctactactgggactggatccgccaggcccccggcaagggcctggagtggatcggcggcttctactactacgagtcctccgagcgcgtgttctacaacccctccctgaagtcccgccccaccgtgtccgtggacacctccaagaaccagttctccctgcgcctgacctccgtgaccgccgccgacaccgccatctactactgcgcccgccgccagttcgactccggcacccccgagggctggttcgacccctggggccagggcaccctggtgaccgtgtcctccggcgacccctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccgcacccccgaggtgacctgcgtggtggtggacgtgtcccacgaggaccccgaggtgaagttcaactggtacgtggacggcgtggaggtgcacaacgccaagaccaagccccgcgaggagcagtacaactccacctaccgcgtggtgtccgtgctgaccgtgctgcaccaggactggctgaacggcaaggagtacaagtgcaaggtgtccaacaaggccctgcccgcccccatcgagaagaccatctccaaggccaagggccagccccgcgagccccaggtgtacaccctgcccccctcccgcgacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccgctggcagcagggcaacgtgttctcctgctccgtgatgcacgaggccctgcacaaccactacacccagaagtccctgtccctgtcccccggcaag

SEQ ID NO: 46 - DNA, 651 bp, Artificial Sequence
  Description of Artificial Sequence Synthetic polynucleotide
cagtccgtgctgacccagcccccctccgtgtccggcgcccccggccagcgcgtgaccatctcctgcaccggctccggctccaacatcggcgccgactacgacgtgcactggtaccagcaggtgcccggcaccgcccccaagctgctgatctacggcgacaccaaccgcccctccggcgtgcccgaccgcttctccggctcccgctccggcacctccgcctccctggtgatcaccggcctgcaggccgaggacgaggccgactactactgccagtcctacgacaacggcctgtccggcttctgggtgttcggcggcggcaccaccctgaccgtgctgggccagcccaaggccgccccctcggtcaccctgttcccgccctcctccgaggagctccaagccaacaaggccacactggtgtgcctcataagcgacttctacccgggagccgtgacagtggcctggaaggcagacagcagccccgtcaaggcgggagtggagaccaccacaccctccaaacaaagcaacaacaagtacgcggccagcagctacctgagcctgacgcccgagcagtggaagtcccacagaagctacagctgccaggtcacgcacgaagggagcaccgtggagaagacagtggcccccacagaatgc

SEQ ID NO: 47 - DNA, 1392 bp, Artificial Sequence
  Description of Artificial Sequence Synthetic polynucleotide
gaggtgcagctggtgcagtccggcgccgaggtgaagaagcccggctcctccgtgaaggtgtcctgcaaggcctccggcgacgccatccgcgaccacgccatctcctgggtgcgccaggcccccggccagggcctggagtggatgggcggcatcgtgtccggcttcggcgccgccaactacgcccagaagttccagggccgcgtgaccatcaccgccgacgactccaccaacaccgtgtacatggaggtgccccgcctgcgctccgacgacaccgccgtgtactactgcgccaagtccgtgtccgccgactcccacgacaagcccaccgtggacttctacttctactacatggacgtgtggggcaccggcaccaccgtgaccgtgtcctccggcgacccctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccgcacccccgaggtgacctgcgtggtggtggacgtgtcccacgaggaccccgaggtgaagttcaactggtacgtggacggcgtggaggtgcacaacgccaagaccaagccccgcgaggagcagtacaactccacctaccgcgtggtgtccgtgctgaccgtgctgcaccaggactggctgaacggcaaggagtacaagtgcaaggtgtccaacaaggccctgcccgcccccatcgagaagaccatctccaaggccaagggccagccccgcgagccccaggtgtacaccctgcccccctcccgcgacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccgctggcagcagggcaacgtgttctcctgctccgtgatgcacgaggccctgcacaaccactacacccagaagtccctgtccctgtcccccggcaag

SEQ ID NO: 48 - DNA, 679 bp, Artificial Sequence
  Description of Artificial Sequence Synthetic polynucleotide
gacatcgtgatgacccagtcccccgactccctggccgtgtccctgggcgagcgcgccaccatcaactgcaagtcctcccagtccatcctgttctccgccctgaacctgaactacctggcctggtaccagcagaagcccggccagcccccccgcctgctgatctactgggcctccacccgcgagtccggcgtgcccgaccgcttctccggctccggctccggcaccgacttcaccctgaccatctcctccctgcaggccgaggacgtggccgtgtactactgccagcagtactacgactccccccccggcctgaccttcggcggcggcacccgcgtggagatcaaagggcgacccctcccgcaccgtggccgccccctccgtgttcatcttccccccctccgacgagcagctgaagtccggcaccgcctccgtggtgtgcctgctgaacaacttctacccccgcgaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccgcggcgagtgc

(19) United States
(12) Patent Application Publication
(10) Pub. No.: US 2022/0340644 A1
(43) Pub. Date: Oct. 27, 2022

(54) INFLUENZA NEUTRALIZING ANTIBODIES AND THEIR USES

(71) Applicant: Duke University, Durham, NC (US)

(72) Inventors: David EASTERHOFF, Durham, NC (US); Gregory SEMPOWSKI, Durham, NC (US)

(21) Appl. No.: 17/761,960

(22) PCT Filed: Sep. 18, 2020
(86) PCT No.: PCT/US2020/051564

§ 371 Date: Mar. 18, 2022

Related U.S. Application Data
(60) Provisional application No. 62/902,705, filed on Sep. 19, 2019.

(51) Int. Cl. C07K 16/10; A61P 31/16

(52) U.S. Cl. CPC C07K 16/1018; A61P 31/16; C07K 2317/52; C07K 2317/565; C07K 2317/622; C07K 2317/55

(57) ABSTRACT

The invention relates to human antibodies binding to and neutralizing Influenza (flu) virus and their uses.

This application claims the benefit of and priority to U.S. Application Ser. No. 62/902,705, filed Sep. 19, 2019 which content is herein incorporated by reference in its entirety.

This invention was made with government support under the Duke DARPA Pandemic Prevention Platform (P3; DoD-DARPA HR0011-17-2-0069) Program. The government has certain rights in the invention.

### SEQUENCE LISTING

The instant application contains a Sequence Listing which has been submitted electronically in ASCII format and is hereby incorporated by reference in its entirety and forms part of the international application. Said ASCII copy, created on Sep. 16, 2020, is named 1234300_00354WO1_SL.txt and is 58,851 bytes in size. The Sequence Listing contains SEQ ID NOs: 1 to 48, which are hereby incorporated by reference.

### TECHNICAL FIELD

The invention relates to human antibodies that bind to and neutralize Influenza (flu) virus and their uses.

### BACKGROUND

Influenza viruses cause annual influenza epidemics and occasional pandemics, which pose a significant threat to public health worldwide. Seasonal influenza infection is associated with 200,000-500,000 deaths each year, particularly in young children, immunocompromised patients and the elderly. Mortality rates typically increase further during seasons with pandemic influenza outbreaks. There remains a significant unmet medical need for potent anti-viral therapeutics for preventing and treating influenza infections, particularly in under-served populations.

There are three types of influenza viruses: types A, B, and C. The majority of influenza disease is caused by influenza A and B viruses (Thompson et al. (2004) JAMA. 292:1333-1340; and Zhou et al. (2012) Clin Infect. Dis. 54:1427-1436). The overall structure of influenza viruses A, B. and C is similar, and includes a viral envelope which surrounds a central core. The viral envelope includes two surface glycoproteins, hemagglutinin (HA) and neuraminidase (NA); HA mediates binding of the virus to target cells and entry into target cells, whereas NA is involved in the release of progeny virus from infected cells.

The HA protein is trimeric in structure and includes three identical copies of a single polypeptide precursor, HAO, which, upon proteolytic maturation, is cleaved into a pH-dependent, metastable intermediate containing a globular head (HA1) and stalk region (HA2) (Wilson et al. (1981) Nature. 289:366-373). The membrane distal globular head constitutes the majority of the HA1 structure and contains the sialic acid binding pocket for viral entry and major antigenic domains.

Influenza A viruses can be classified into subtypes based on genetic variations in hemagglutinin (HA) and neuraminidase (NA) genes. Currently, in seasonal epidemics, influenza A H1 and H3 HA subtypes are primarily associated with human disease, whereas viruses encoding H5, H7, H9 and H10 are associated with sporadic human outbreaks due to direct transmission from animals.

In contrast to influenza A viruses, influenza B viruses are not divided into subtypes based on the two surface glycoproteins and until the 1970s were classified as one homogenous group. Through the 1970s, the influenza B viruses started to diverge into two antigenically distinguishable lineages which were named the Victoria and Yamagata lineages after their first representatives, B/Victoria/2/87 and B/Yamagata/16/88, respectively. (Biere et al. (2010) J Clin Microbiol. 48(4):1425-7; doi: 10.1128/JCM.02116-09. Epub Jan. 27, 2010). Influenza B viruses are restricted to human infection, and both lineages contribute to annual epidemics. Although the morbidity caused by influenza B viruses is lower than that associated with influenza A H3N2, it is higher than that associated with influenza A H1N1 (Zhou et al. (2012) Clin Infect. Dis. 54:1427-1436).

### SUMMARY OF THE INVENTION

In certain aspects, the invention provides a recombinant antibody or antigen-binding fragment thereof comprising VLCDR1, VLCDR2, VLCDR3 and VHCDR1, VHCDR2, VHCDR3 (FIG. 5-10, 11, or 12) of the respective antibodies in Table 3.

In certain aspects, the invention provides a recombinant antibody or antigen-binding fragment thereof comprising the VL and VH (FIG. 5-10, 11 or 12) of the antibodies in Table 3.

In certain aspects, the invention provides a pharmaceutical composition comprising the recombinant antibodies or antigen-binding fragments thereof of the invention.

In certain aspects, the invention provides nucleic acids comprising sequences encoding flu antibodies or antigen-binding fragment thereof comprising VLCDR1, VLCDR2, VLCDR3 and VHCDR1, VHCDR2, VHCDR3 sequences of the invention. In certain aspects, the invention provides nucleic acids comprising sequences encoding flu antibodies or antigen-binding fragment thereof comprising VL and VH sequences of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies or antigen-binding fragment thereof. In certain embodiments, these are optionally formulated in lipid nanoparticles (LNPs). In certain embodiments, the mRNAs are modified. Modifications include without limitations modified ribonucleotides, poly-A tail, 5′ cap.

In certain aspects, the invention provides prophylactic methods comprising administering the pharmaceutical composition of the invention. In certain embodiments, the methods lead to protection from disease, reduced severity of disease, including but not limited to reduced severity of symptoms and/or reduced duration of flu disease.

In certain aspects, the invention provides methods of treatment comprising administering the pharmaceutical composition of the invention.

In certain embodiments, the methods comprise administering additional therapeutic or prophylactic agents, including but not limited to additional flu antibodies or antigen-binding fragment thereof, small molecule therapeutics, or any other suitable agent. In certain embodiments, the flu antibodies or antigen-binding fragment thereof administered have different specificities.

In certain aspects, the invention provides a kit comprising: a composition comprising an antibody or antigen-binding fragment thereof of the invention, a syringe, needle, or applicator for administration of the antibody to a subject; and instructions for use.

In certain aspects the invention provides a method of treating a subject, the method comprising steps of: administering to a subject suffering from or susceptible to influenza infection therapeutically effective amount of an antibody or antigen-binding fragment thereof of the invention.

In certain aspects, the invention provides a recombinant influenza antibody, or an antigen-binding fragment thereof, wherein in certain non-limiting embodiments the antibody or antigen-binding fragment thereof specifically binds to an influenza virus and neutralizes influenza virus infection. In certain embodiments, the antibody specifically binds to an influenza epitope. In certain embodiments, the antibody, or the antigen-binding fragment thereof, wherein the concentration of the antibody, or antigen-binding fragment thereof, required for 50% neutralization of influenza virus (IC50) is as described in Example 1. In certain embodiments, the IC50 is up to about 1 microgram/ml, up to about 500 ng/ml, up to about 250 ng/ml, up to about 100 ng/ml or up to about 50 ng/ml.

In certain embodiments, the antibody, or the antigen-binding fragment thereof, according to the invention binds to H3N2 virus. In certain embodiments the virus is as described in FIG. 2 or FIG. 13.

In certain embodiments, the antibody, or antigen-binding fragment thereof are H3 HA specific.

In certain embodiments, the antibody or antigen-binding fragment thereof, is a fully human antibody.

In certain embodiments, the antibody, or antigen-binding fragment thereof, is a recombinant human monoclonal antibody.

In certain embodiments, the antibody, or antigen-binding fragment thereof, comprises an Fc moiety. In certain embodiments, the antibody, or antigen-binding fragment thereof, comprises a mutation(s) in the Fc moiety, in certain embodiments the mutation reducing binding of the antibody to an Fc receptor, in certain embodiments the mutation increasing the half-life of the recombinant antibody. In certain embodiments, the Fc mutation is LS mutation.

In certain embodiments, the recombinant influenza antibody or the antigen-binding fragment thereof, is described in Example 1, FIG. 14, or FIGS. 5-10.

In certain embodiments, the antibody or antigen-binding fragment thereof, comprises a heavy chain (VH) comprising at least one CDRH1, at least one CDRH2 and at least one CDRH3 and a light chain (VL) comprising at least one CDRL1, at least one CDRL2 and at least one CDRL3, wherein at least one CDR, comprises, consists essentially of, or consists of an amino acid sequence according to any of the sequences listed in FIG. 5-10, or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional variation is 80%, 81%, 82%, 83%, 84%, 85%, 86%, 87%, 88%, 89%, 90%, 91%, 92%, 93%, 94%, 95%, 96%, 97%, 98% or 99% identity to any of the sequences listed in FIGS. 5-10. In certain embodiments, the CDRs are selected from the VH and VL sequence of Ab700364.

In certain embodiments, the antibody or antigen-binding fragment thereof, comprises a heavy chain comprising at least one CDRH11, at least one CDRH12 and at least one CDRH13 and a light chain comprising at least one CDRL1, at least one CDRL2 and at least one CDRL3, wherein at least one CDR, comprises, consists essentially of, or consists of an amino acid sequence according to any of the sequences of VH H700364 or of VL K7000263 in FIGS. 5A and 5B, or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional variation is 80%, 81%, 82%, 83%, 84%, 85%, 86%, 87%, 88%, 89%, 90%, 91%, 92%, 93%, 94%, 95%, 96%, 97%, 98% or 99% identity.

In certain embodiments, the antibody or antigen-binding fragment thereof, comprises a heavy chain comprising CDRH1, CDRH12, and CDRH3 and a light chain comprising CDRL1, CDRL2, and CDRL3, wherein at least one CDR, comprises, consists essentially of, or consists of an amino acid sequence according to any of the sequences of VH H700364 or of VL K7000263 in FIGS. 5A and 5B, or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional variation is 80%, 81%, 82%, 83%, 84%, 85%, 86%, 87%, 88%, 89%, 90%, 91%, 92%, 93%, 94%, 95%, 96%, 97%, 98% or 99% identity.

In certain embodiments, the antibody or antigen-binding fragment thereof, comprises, consists essentially of, or consists of a VH amino acid sequence VH H700364 or a VL amino acid sequence VL K7000263 in FIGS. 5A and 5B or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments the functional variation is 80%, 81%, 82%, 83%, 84%, 85%, 86%, 87%, 88%, 89%, 90%, 91%, 92%, 93%, 94%, 95%, 96%, 97%, 98% or 99% identity. In certain embodiments, the functional variation is outside of the CDRs.

In certain embodiments, the antibody or antigen-binding fragment thereof, comprises, consists essentially of, or consists of a VH amino acid sequence according to VH H700364 or a VL amino acid sequence according to VL K7000263 in FIGS. 5A and 5B. In certain embodiments, the antibody or antigen binding fragment thereof, comprises, consists essentially of, or consists of a VH amino acid sequence according to VH H700364 and a VL amino acid sequence according to VL K7000263 in FIGS. 5A and 5B. In certain embodiments, the antibody is Ab700364.

In certain embodiments, the antibody, or the antigen-binding fragment thereof, is a purified antibody, a single chain antibody, Fab, Fab′, F(ab′)2, Fv or scFv.

In certain embodiments, the antibody any isotype.

In certain embodiments the antibody or antigen-binding fragment thereof of the invention is formulated for use as a medicament.

In certain embodiments the antibody or antigen-binding fragment thereof of the invention is formulated for use in the prevention and/or treatment of influenza virus infection.

In certain aspects, the invention provides a nucleic acid molecule comprising a polynucleotide encoding the antibody, or the antigen-binding fragment thereof, according to the invention. In non-limiting embodiments, the nucleic acid is optimized for expression in a suitable host cell. In non-limiting embodiments, the nucleic acid is optimized for in vitro expression as an mRNA therapeutic molecule.

In certain embodiments, the polynucleotide sequence comprises, consists essentially of, or consists of a nucleic acid sequence according to any one of the sequences in FIG. 5-10; or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional variation is 80%, 81%, 82%, 83%, 84%, 85%, 86%, 87%, 88%, 89%, 90%, 91%, 92%, 93%, 94%, 95%, 96%, 97%, 98% or 99% identity. In certain embodiments, the antibody is Ab700364. In certain embodiments, the antibody is Ab700446.

In certain embodiments, the nucleic acid is a ribonucleic acids (RNA) as shown in FIGS. 11A-11L. In certain embodiments, the RNA is mRNA which suitable for use and delivery as a therapeutic mRNA. In certain embodiments, the mRNA comprises a 5′-terminal CAP modification. In certain embodiments, the mRNA comprises modified nucleotides. In certain embodiments, the mRNA is formulated in a lipid nanoparticle.

In certain aspects, the invention provides a vector comprising a nucleic acid molecule encoding an antibody or antigen-binding fragment thereof of the invention.

In certain aspects, the invention provides a cell expressing the antibody, or the antigen-binding fragment thereof, of the invention. In certain embodiments, the cell comprises a vector comprising a nucleic acid molecule encoding an antibody antigen-binding fragment thereof of the invention.

In certain aspects, the invention provides a pharmaceutical composition comprising the antibody, or the antigen-binding fragment thereof of the invention. In certain aspects, invention provides a pharmaceutical composition comprising a nucleic acid sequence of the invention, a vector comprising nucleic acid sequence of the invention and/or a cell comprising a nucleic acid or vector of the invention. In certain embodiments, the pharmaceutical composition optionally comprises a pharmaceutically acceptable excipient, diluent or carrier.

In certain aspects, the invention provides a method of treating or preventing influenza infection in a subject in need thereof, comprising administering the recombinant influenza antibody or antigen-binding fragment thereof of the invention, a nucleic acid encoding an antibody or antigen-binding fragment thereof of the invention, or a pharmaceutical composition comprising these, in an amount suitable to effect treatment or prevention of influenza infection.

In non-limiting embodiments of the methods, administering is prior to influenza exposure or at the same time as influenza exposure.

In certain aspects, the invention provides an in vitro transcription system to synthesize ribonucleic acids (RNAs) encoding antibodies or antigen-binding fragment thereof of the invention, comprising: a reaction vessel, a DNA vector template comprising nucleic acid sequence encoding an antibody or antigen-binding fragment thereof of the invention as described herein, and reagents for carrying out an in vitro transcription reaction that produces mRNA encoding an antibody or antigen-binding fragment thereof of the invention. In certain embodiments, the mRNA is modified mRNA.

In certain aspects, the invention provides a method for manufacturing an mRNA encoding an antibody or antigen-binding fragment thereof, comprising:
a. providing an in vitro transcription reaction vessel comprising a DNA template encoding an antibody or antigen-binding fragment thereof according to any of the preceding claims and reagents under conditions suitable for in vitro transcription of the nucleic acid template, thereby producing an mRNA template encoding the antibody or antigen-binding fragment thereof according to any of the preceding claims, and
b. isolating the mRNA by any suitable method of purification and separating reaction reagents, the DNA template, and/or mRNA product related impurities. In certain embodiments, the mRNA comprises modified nucleotides. In certain embodiments, the mRNA comprises 5′-CAP, and/or any other suitable modification.

In certain aspects, the invention provides a method for manufacturing an antibody or antigen-binding fragment thereof, comprising culturing a host cell comprising a nucleic acid according to any of the preceding claims under conditions suitable for expression of the antibody or antigen-binding fragment thereof and isolating said antibody or antigen-binding fragment thereof.

### BRIEF DESCRIPTION OF DRAWINGS

The patent or application file contains at least one drawing executed in color. To conform to the requirements for PCT patent applications, many of the figures presented herein are black and white representations of images originally created in color.

FIG. 1 shows that Live/CD3 neg/CD14 neg/CD16 neg/16 neg/CD19 pos/fluorescent whole flu virus-specific B cells were single cell sorted into PCR plates.

FIG. 2 shows antibody binding to a panel of HA proteins derived from H3N2, H1N1, H5N1, and H7N9 influenza viruses. Antibodies were assayed for binding by ELISA.

FIG. 3 shows in vivo antibody-mediated protection study with antibody delivered as protein. In vivo antibody-mediated protection study with antibody delivered as protein and A/Aichi/2/1968 X-31 virus challenge. Mice that received no antibody (N=8), intramuscular delivery of 20 mg/kg of Ab700364, Ab700446 or Ab700438 (N=10) or intranasal co-administration of Ab700364+ virus were challenged with 1×106 FFU of A/Aichi/2/1968 X-31 virus. Shown are the survival curves for each group. Ab700364 delivered as a protein 24 hours prior to challenge or co-administered intranasal with the virus at the time of challenge provided 100% protection from A/Aichi/2/1968 X-31 infection related death. At 7 days post challenge 100% of mice receiving Ab700446 died and 80% Ab700438 died.

FIG. 4 shows in vivo antibody-mediated protection study with antibody delivered as mRNA-LNP. In vivo antibody-mediated protection study with antibody delivered as mRNA-LNP and A/Aichi/2/1968 X-31 virus challenge. Mice that received no antibody (N=21), intravenous delivery of 35 μgs of Ab700364, Ab700446 or Ab700438 modified mRNA-LNP (N=10 per group). Shown are the survival curves for each group. Mice immunized with miRNA LNPs encoding Ab700358 and Ab700364 were 100% protected from A/Aichi/2/1968 X-31 infection related death when challenged with virus either 24 hours or 72 hours after mRNA-LNP delivery. Mice receiving Ab700446 were partially protected at both 24 hours and 72 hours.

FIGS. 5A-5B show maps of sequences of VH chains of lineage Ab700364. FIG. 5A shows non-limiting embodiments of the Ab700364 Clonal Lineage heavy chains nucleic acid sequences. FIG. 5B shows the Ab700364 Clonal Lineage heavy chains amino acid sequences. FIGS. 5A-B disclose SEQ ID NOS 1-8, respectively, in order of appearance.

FIGS. 6A-613 show maps of sequences of VL chains of lineage Ab700364. FIG. 6A shows non-limiting embodiments of the Ab700364 Clonal Lineage light chains nucleic acid sequences. FIG. 6B shows the Ab700364 Clonal Lineage light chains amino acid sequences. FIGS. 6A-B disclose SEQ ID NOS 9-16, respectively, in order of appearance.

FIGS. 7A-7B show sequences of antibodies of the invention. FIG. 7A shows non-limiting embodiments of the Ab700451 Clonal Lineage heavy chain nucleic acid sequence. FIG. 7B shows the Ab700451 Clonal Lineage heavy chain amino acid sequence. FIGS. 7A-B disclose SEQ ID NOS 17-18, respectively, in order of appearance.

FIGS. 8A-8B show sequences of antibodies of the invention. FIG. 8A shows non-limiting embodiments of the Ab700451 Clonal Lineage light chain nucleic acid sequence. FIG. 8B shows the Ab700451 Clonal Lineage light chain amino acid sequence. FIGS. 8A-B disclose SEQ ID NOS 19-20, respectively, in order of appearance.

FIGS. 9A-9B show sequences of antibodies of the invention. FIG. 9A shows non-limiting embodiments of the Ab700458 Clonal Lineage heavy chain nucleic acid sequence. FIG. 9B shows the Ab700458 Clonal Lineage heavy chain amino acid sequence. FIGS. 9A-B disclose SEQ ID NOS 21-22, respectively, in order of appearance.

FIGS. 10A-10B show sequences of antibodies of the invention. FIG. 10A shows non-limiting embodiments of the Ab700458 Clonal Lineage light chain nucleic acid sequence. FIG. 10B shows the Ab700458 Clonal Lineage light chain amino acid sequence. FIGS. 10A-B disclose SEQ ID NOS 23-24, respectively, in order of appearance.

FIGS. 11A-L show non-limiting embodiments of RNA sequences of antibodies of the invention. FIGS. 11A-L disclose SEQ ID NOS 25-36, respectively, in order of appearance.

FIG. 12 shows non-limiting embodiments of DNA sequence of antibodies of the invention. FIG. 12 discloses SEQ ID NOS 37-48, respectively, in order of appearance.

FIG. 13 shows in vitro neutralization of A/Hong Kong/7/4801/2014 and A/Aichi/2/1968 X-31 by P3-isolated antibodies isolated with fluorescently labeled A/Hong Kong/7/4801/2014 virus. Shown are the minimum effective neutralization concentrations (μg/mL).

FIG. 14 shows immunogenetics of A/Hong Kong/7/4801/2014 neutralizing antibodies isolated by fluorescent whole virus sorting. Sanger sequencing AB1 files were analyzed by Cloanalyst.

FIG. 15 shows in vivo pharmacokinetics of mRNA/LNP Flu countermeasure in Non-human Primates.

### DETAILED DESCRIPTION

The World Health Organization (WHO) estimates that influenza transmission results in up to 500,000 deaths annually. Furthermore, influenza virus has been estimated to be responsible for an annual economic burden in excess of $87 billion. The current standard of care for influenza prevention is seasonal immunization with a multivalent cocktail of inactivated influenza viruses predicted to be antigenically representative of circulating strains (H1N1. H3N2 and B). The majority of influenza virus vaccine preparations available for human vaccination in the United States are generated via propagation in embryonated eggs using a procedure that has remained virtually unchanged since the mid-1900's. The generation/administration of vaccines in this way 1) is laborious resulting in a minimum five to six-month lag between virus isolation and vaccine availability 2) may select for egg-adaptation mutations resulting in a loss of immunogenicity with regard to circulating strains, and 3) requires sufficient time post-immunization for the development of virus-specific antibodies (˜2 weeks). Thus, there is a critical need for fast-acting antiviral countermeasures, such as therapeutic and/or prophylactic antibodies, that can be rapidly isolated, evolved, manufactured, and safely delivered to at risk individuals. In non-limiting embodiments, the invention provides such flu antibodies.

Production of Antibodies

Antibodies or antigen-binding fragment thereof according to the invention can be made by any method known in the art.

In certain embodiments, plasma cells are cultured in limited numbers, or as single plasma cells in microwell culture plates. Antibodies can be isolated from the plasma cell cultures. VH and VL can be isolated from single cell sorted plasma cells. From the plasma cell, RNA can be extracted and PCR can be performed using methods known in the art. The VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

In some aspects, recombinant antibodies of the invention comprise antibodies produced by amplifying Ig genes and expressing these sequences in any suitable host cell.

Fragments of the antibodies of the invention can be obtained from the antibodies by methods that include digestion with enzymes, such as pepsin or papain, and/or by cleavage of disulfide bonds by chemical reduction. Alternatively, fragments of the antibodies can be obtained by cloning and expression of part of the sequences of the heavy or light chains. Antibody “fragments” include Fab, Fab′, F(ab′)2 and Fv fragments. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and diners, single domain heavy chain antibodies, single domain light chain antibodies, as well as single chain antibodies, e.g., single chain Fv in which the heavy and light chain variable domains are joined by a peptide linker.

Antibody fragments of the invention may impart monovalent or multivalent interactions and be contained in a variety of structures as described above. For instance, scFv molecules may be synthesized to create a trivalent “triabody” or a tetravalent “tetrabody.” The scFv molecules may include a domain of the Fc region resulting in bivalent minibodies. In addition, the sequences of the invention may be a component of multispecific molecules in which the sequences of the invention target the epitopes of the invention and other regions of the molecule bind to other targets. Exemplary molecules include, but are not limited to, bispecific Fab2, trispecific Fab3, bispecific scFv, and diabodies (Holliger and Hudson, 2005, Nature Biotechnology 9: 1126-1136).

Standard techniques of molecular biology may be used to prepare DNA sequences encoding the antibodies or antibody fragments of the present invention. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention or fragments thereof. Bacterial, for example E. coli, and other microbial systems may be used, in part, for expression of antibody fragments such as Fab and F(ab′)2 fragments, and especially Fv fragments and single chain antibody fragments, for example, single chain Fvs. Eukaryotic, e.g., mammalian, host cell expression systems may be used for production of larger antibody molecules, including complete antibody molecules. Suitable mammalian host cells include, but are not limited to, CHO, HEK293T, PER.C6, NS0, myeloma or hybridoma cells. Mammalian cell lines suitable for expression of therapeutic antibodies are well known in the art.

The present invention also provides a process for the production of an antibody molecule according to the present invention comprising culturing a host cell comprising a vector encoding a nucleic acid of the present invention under conditions suitable for expression of protein from DNA encoding the antibody molecule of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides. Alternatively, antibodies according to the invention may be produced by (i) expressing a nucleic acid sequence according to the invention in a host cell, e.g. by use of a vector according to the present invention, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

B cell clones or transfected host-cells of the invention can be used in various ways e.g., as a source of monoclonal antibodies, as a source of nucleic acid (DNA or mRNA) encoding a monoclonal antibody of interest, for research, etc.

Expression from recombinant sources is more common for pharmaceutical purposes than expression from B cells or hybridomas e.g., for reasons of stability, reproducibility, culture ease, etc.

Thus the invention also provides a method for preparing a recombinant cell, comprising the steps of: (i) obtaining one or more nucleic acids (e.g., heavy and/or light chain mRNAs) from a B cell clone or cultured plasma cells that encodes the antibody of interest; (ii) inserting the nucleic acid into an expression vector and (iii) transfecting the vector into a host cell in order to permit expression of the antibody of interest in that host cell.

Similarly, the invention provides a method for preparing a recombinant cell, comprising the steps of: (i) sequencing nucleic acid(s) from a B cell clone or cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest, wherein the nucleic acids are nucleic acids that were derived from a cell sorted B cell or a cultured plasma cell of the invention.

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including, but not limited to, static culture, roller bottle culture, ascites fluid, hollow-fiber type bioreactor cartridge, modular minifermenter, stirred tank, microcarrier culture, ceramic core perfusion, etc.

Any suitable host cells could be used for transfection and production of the antibodies of the invention. The transfected host cell may be a eukaryotic cell, including yeast and animal cells, particularly mammalian cells (e.g., CHO cells, NS0 cells, human cells such as PER.C6 or HKB-11 cells, myeloma cells, or a human liver cell), as well as plant cells. In certain embodiments, expression hosts can glycosylate the antibody of the invention, particularly with carbohydrate structures that are not themselves immunogenic in humans. In one embodiment, the transfected host cell may be able to grow in serum-free media. In a further embodiment, the transfected host cell may be able to grow in culture without the presence of animal-derived products. The transfected host cell may also be cultured to give a cell line.

In certain aspects the invention provides nucleic acids encoding the inventive influenza antibodies or antigen-binding fragments thereof. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

Pharmaceutical Composition

The present invention also provides a pharmaceutical composition comprising one or more of: (i) the antibody, or the antigen-binding fragment thereof, according to the present invention; (ii) the nucleic acid encoding the antibody, or antigen-binding fragments according to the present invention; (iii) the vector comprising the nucleic acid according to the present invention; and/or (iv) the cell expressing the antibody or antigen-binding fragment thereof according to the present invention or comprising the vector according to the present invention.

In certain aspects, the invention provides a pharmaceutical composition comprising the antibody, or the antigen-binding fragment thereof, according to the present invention, the nucleic acid according to the present invention, the vector according to the present invention and/or the cell according to the present invention.

The pharmaceutical composition may also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In general, pharmaceutically acceptable carriers in a pharmaceutical composition according to the present invention may be active components or inactive components. In certain embodiments the pharmaceutically acceptable carrier in a pharmaceutical composition according to the present invention is not an active component in respect to flu virus infection.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol, and ethanol. Additionally, auxiliary substances, such as wetting or emulsifying agents or pH buffering substances, may be present in such compositions. Such carriers enable the pharmaceutical compositions to be formulated as tablets, pills, capsules, liquids, gels, syrups, slurries and suspensions, for ingestion by the subject.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin™, for reconstitution with sterile water containing a preservative). The composition may be prepared for topical administration e.g., as an ointment, cream, or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody or antigen-binding fragment thereof may be provided in kit form with sterile water or a sterile buffer.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH between 5.5 and 8.5, in some embodiments this may be between 6 and 8, and in other embodiments about 7. The pH may be maintained by the use of a buffer. The composition may be sterile and/or pyrogen free. The composition may be isotonic with respect to humans. In one embodiment pharmaceutical compositions of the invention are supplied in hermetically-sealed containers.

Within the scope of the invention are compositions present in several forms of administration: the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody molecule or antigen-binding fragment thereof may be in dry form, for reconstitution before use with an appropriate sterile liquid. A vehicle is typically understood to be a material that is suitable for storing, transporting, and/or administering a compound, such as a pharmaceutically active compound, in particular the antibodies or antigen-binding fragments thereof according to the present invention. For example, the vehicle may be a physiologically acceptable liquid, which is suitable for storing, transporting, and/or administering a pharmaceutically active compound, in particular the antibodies or antigen-binding fragments thereof according to the present invention. Once formulated, the compositions of the invention can be administered directly to the subject. In one embodiment the compositions are adapted for administration to mammalian, e.g., human subjects.

The pharmaceutical compositions of this invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the invention. In certain embodiments, the pharmaceutical composition may be prepared for oral administration, e.g. as tablets, capsules and the like, for topical administration, or as injectable, e.g. as liquid solutions or suspensions. In certain embodiments, the pharmaceutical composition is an injectable. Solid forms suitable for solution in, or suspension in, liquid vehicles prior to injection are also contemplated, e.g. that the pharmaceutical composition is in lyophilized form.

For injection, e.g. intravenous, cutaneous or subcutaneous injection, or injection at the site of affliction, the active ingredient could be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is a polypeptide, peptide, or nucleic acid molecule, other pharmaceutically useful compound according to the present invention that is to be given to an individual, administration is in a “prophylactically effective amount” or a “therapeutically effective amount,” this being sufficient to show benefit to the individual. The actual amount administered, and rate and time-course of administration, will depend on the nature and severity of what is being treated. For injection, the pharmaceutical composition according to the present invention may be provided for example in a pre-filled syringe.

The inventive pharmaceutical composition as defined above may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the inventive transporter cargo conjugate molecule as defined above, is combined with emulsifying and suspending agents. If desired, certain sweetening, flavoring or coloring agents may also be added.

The inventive pharmaceutical composition may also be administered topically, especially when the target of treatment includes areas or organs readily accessible by topical application, e.g. including diseases of the skin or of any other accessible epithelial tissue. Suitable topical formulations are readily prepared for each of these areas or organs. For topical applications, the inventive pharmaceutical composition may be formulated in a suitable ointment, containing the inventive pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the inventive pharmaceutical composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. In particular, the pharmaceutical composition may be provided as single-dose product. In certain embodiments, the amount of the antibody or antigen-binding fragment thereof in the pharmaceutical composition—in particular if provided as single-dose product—does not exceed 200 mg. In certain embodiments, the amount does not exceed 100 mg, and in certain embodiments, the amount does not exceed 50 mg.

In non-limiting embodiments, the antibodies or antigen-binding fragments thereof of the invention could be used for non-therapeutic uses, such as but not limited to diagnostic assays.

Sequence Variants and Identity

Sequence identity is usually calculated with regard to the full length of the reference sequence (i.e. the sequence recited in the application). Percentage identity, as referred to herein, can be determined, for example, using BLAST using the default parameters specified by the NCBI (the National Center for Biotechnology Information; www.ncbi.nlm.nih.gov) [Blosum 62 matrix; gap open penalty=11 and gap extension penalty=1].

Fc

In some embodiments, the nucleic acid encoding the VH and VL can encode an Fc domain (immunoadhesin). The Fc domain can be an IgA, IgM or IgG Fc domain. The Fc domain can be an optimized Fc domain, as described in U.S. Published Patent Application No. 20100093979, incorporated herein by reference. In one example, the immunoadhesin is an IgG1 Fc. In one example, the immunoadhesin is an IgG3 Fc.

In one embodiment, the IgG constant region comprises the LS mutation. Additional variants of the Fc portion of the antibody are also contemplated by the invention. See Maeda et al. MAbs. 2017 July; 9(5): 844-853. Published online Apr. 7, 2017, PMID: 28387635; see also Booth et al. MAbs. 2018 October; 10(7): 1098-1110. Published online Jul. 26, 2018. doi: 10.1080/19420862.2018.1490119.

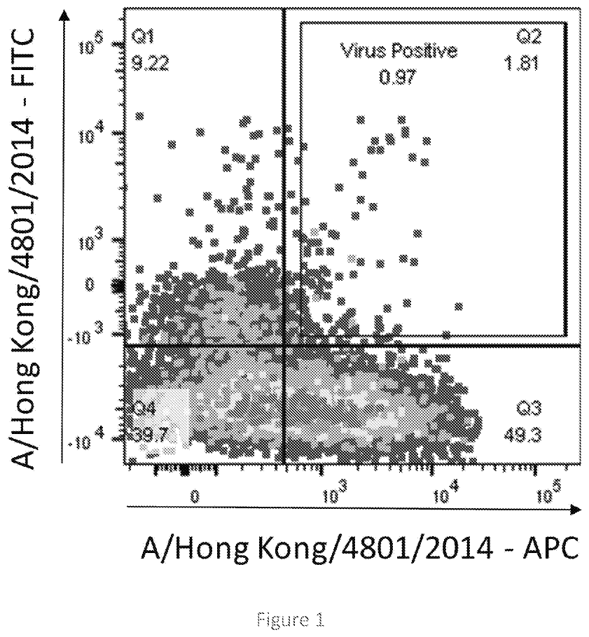

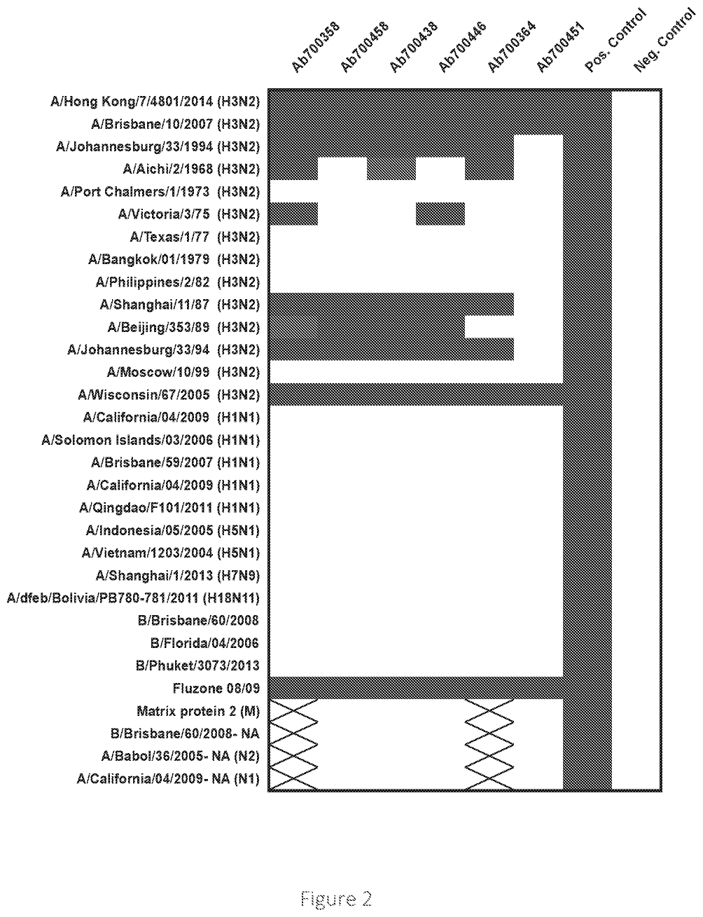

In certain embodiments, the antibodies comprise amino acid alterations, or combinations thereof, for example in the Fc region outside of epitope binding, which alterations can improve their properties. Various Fc modifications are known in the art. Amino acid numbering regarding Fc modification is according to the EU Index in Kabat. In some embodiments, the invention contemplates antibodies comprising mutations that affect neonatal Fc receptor (FcRn) binding, antibody half-life, and localization and persistence of antibodies at mucosal sites. See e.g. Ko S Y et al., Nature 514: 642-45, 2014, at FIG. 1a and citations therein; Kuo, T. and Averson, V., mAbs 3(5): 422-430, 2011, at Table 1, U.S. Pub 20110081347 (an aspartic acid at Kabat residue 288 and/or a lysine at Kabat residue 435), U.S. Pub 20150152183 for various Fc region mutation, each of which references are incorporated by reference in their entireties. In certain embodiments, the antibodies comprise AAAA substitution in and around the Fc region of the antibody that has been reported to enhance ADCC via NK cells (AAA mutations) containing the Fc region aa of S298A as well as E333A and K334A (Shields R I et al J B C, 276: 6591-6604, 2001) and the 4^{th} A (N434A) is to enhance FcR neonatal mediated transport of the IgG to mucosal sites (Shields R I et al. ibid). Other antibody mutations have been reported to improve antibody half-life or function or both and can be incorporated in sequences of the antibodies. These include the DLE set of mutations (Romain G, et al. Blood 124: 3241, 2014), the LS mutations M428L/N4345, alone or in a combination with other Fc region mutations, (Ko S Y et al. Nature 514: 642-45, 2014, at FIG. 1a and citations therein: Zlevsky et al., Nature Biotechnology, 28(2): 157-159, 2010; U.S. Pub 20150152183); the YTE Fc mutations (Robbie G et al Antimicrobial Agents and Chemotherapy 12: 6147-53, 2013) as well as other engineered mutations to the antibody such as QL mutations, IHH mutations (Ko S Y et al. Nature 514: 642-45, 2014, at FIG. 1a and relevant citations; See also Rudicell R et al. J. Virol 88: 12669-82, 201). In some embodiments, modifications, such as but not limited to antibody fucosylation, may affect interaction with Fc receptors (See e.g. Moldt et al. J V I 86(11): 66189-6196, 2012). In some embodiments, the antibodies can comprise modifications, for example but not limited to glycosylation, which reduce or eliminate polyreactivity of an antibody. See e.g. Chuang, et al. Protein Science 24: 1019-1030, 2015. In some embodiments the antibodies can comprise modifications in the Fc domain such that the Fc domain exhibits, as compared to an unmodified Fc domain enhanced antibody dependent cell mediated cytotoxicity (ADCC); increased binding to FcγRIIA or to FcγRIIIA; decreased binding to FcγRIIB; or increased binding to FcγRIIB. See e.g. U.S. Pub 20140328836.

In certain embodiments, the antibody, or antigen-binding fragment thereof, comprises a CH2 L4A mutation, a CH2 L5A mutation, or both. See U.S. Patent Publication 20190256582 incorporated by reference in its entirety.

CDRs and Frameworks

In some embodiments, the CDRs of the antibodies and antigen-binding fragments thereof of the invention are defined according to the IMGT scheme. IMGT-defined CDR regions have been highlighted/underlined in the nucleotide and amino acid sequences for each of the VH and VL variable regions of the antibodies of the invention. See Example 1. The IMGT sequence analysis tools will identify CDR and framework regions in the nucleotide sequence and translated amino acid sequence. See www.imgt.org/IMGT_vquest/analysis.

In some embodiments, CDR and framework regions can be identified based on other classical variable region numbering and definition schemes or conventions, including the Kabat, Chothia, Martin, and Aho schemes. The ANARCI (Antigen receptor Numbering And Receptor Classification; see http://opig.stats.ox.ac.uk/webapps/newsabdab/sabpred/anarci/) online tool allows one to input amino acid sequences and to select an output with the IMGT, Kabat, Chothia, Martin, or AHo numbering scheme. With these numbering schemes, CDR and framework regions within the amino acid sequence can be identified. The person of ordinary skill is able to ascertain CDR and framework boundaries using one or more of several publicly available tools and guides.

For example, Table 1 below provides a general, not limiting guide, for the CDR regions as based on different numbering schemes (see www.bioinf.org.uk/abs/info.html #cdrid). In the Table, any of the numbering schemes can be used for these CDR definitions, except the Contact CDR definition uses the Chothia or Martin (Enhanced Chothia) numbering.

TABLE 1
General Guide of Selected CDR Definitions

| | Kabat CDR | AbM CDR | Chothia CDR | Contact CDR | IMGT CDR |
|---|---|---|---|---|---|
| CDR Loop | Definition | Definition | Definition | Definition | Definition |
| CDRL1 | L24-L34 | L24-L34 | L24-L34 | L30-L36 | L27-L32 |
| CDRL2 | L50-L56 | L50-L56 | L50-L56 | L46-L55 | L50-L51 |
| CDRL3 | L89-L97 | L89-L97 | L89-L97 | L89-L96 | L89-L97 |
| CDRH1 | H31-H35B | H26-H35B | H26-H32 . . . | H30-H35B | H26-H35B |
| (Kabat | | | H34 | | |
| numbering) | | | | | |
| CDRH1 | H31-H35 | H26-H35 | H26-H32 | H30-H35 | H26-H33 |
| (Chothia | | | | | |
| numbering) | | | | | |
| CDRH2 | H50-H65 | H50-H58 | H52-H56 | H47-H58 | H51-H56 |
| CDRH3 | H95-H102 | H95-H102 | H95-H102 | H93-H101 | H93-H102 |

In Table 1 above, for CDRH1 Kabat numbering using the Chothia CDR definition, the boundary is H26 to H32 or H34 because the Kabat numbering convention varies between H32 and H34 depending on the length of the loop. This is because the Kabat numbering scheme places insertions at H35A and H35B. If neither H35A nor H35B is present, CDRH42 ends at H32. If only H35A is present, the loop ends at H33. If both H35A and H35B are present, the loop ends at H34.

Alternatively or in combination, one can examine amino acid sequences and identify CDR and framework regions according to the following alternative general guideline of Table 2, which is non-limiting.

TABLE 2
General Guide for Demarcating CDR Boundaries

| CDR Region | Guidelines |
|---|---|
| LCDR1 | Start: approx. residue 24 |
| | Residue before: always a Cys |
| | Residue after: always a Trp, Typically Trp-Tyr-Gln, |
| | but also, Trp-Leu-Gln, Trp-Phe-Gln, Trp-Tyr-Leu |
| | Length: 10 to 17 residues |
| LCDR2 | Start: always 16 residues after the end of L1. |
| | Residues before generally Ile-Tyr, but also, |
| | Val-Tyr, Ile-Lys, Ile-Phe |
| | Length: always 7 residues (except NEW (7FAB) which |
| | has a deletion in this region) |
| LCDR3 | Start: always 33 residues after end of L2 (except NEW |
| | (7FAB) which has the deletion at the end of CDR-L2). |
| | Residue before always Cys. |
| | Residues after |
| | always Phe-Gly-XXX-Gly |
| | Length |
| | 7 to 11 residues |
| HCDR1 | Start |
| | Approx. residue 26 (always 4 after a Cys) [Chothia/AbM |
| | definition]; |
| | Kabat definition starts 5 residues later |
| | Residues before |
| | always Cys-XXX-XXX-XXX |
| | Residues after |
| | always a Trp. Typically Trp-Val, but also, Trp-Ile, |
| | Trp-Ala |
| | Length |
| | 10 to 12 residues [AbM definition]: |
| | Chothia definition excludes the last 4 residues |
| HCDR2 | Start |
| | always 15 residues after the end of Kabat/AbM definition) |
| | of CDR-H1 |
| | Residues before |
| | typically Leu-Glu-Trp-Ile-Gly, but a number of variations |
| | Residues after |
| | Lys/Arg-Leu/Ile/Val/Phe/Thr/Ala-Thr/Ser/Ile/Ala |
| | Length |
| | Kabat definition 16 to 19 residues; |
| | AbM (and recent Chothia) definition ends 7 residues earlier |
| HCDR3 | Start |
| | always 33 residues after end of CDR-H2 (always 2 after |
| | a Cys) |
| | Residues before |
| | always Cys-XXX-XXX (typically Cys-Ala-Arg) |
| | Residues after |
| | always Trp-Gly-XXX-Gly |
| | Length |
| | 3 to 25(!) residues |

In the instant figures, some CDR boundaries are underlined and these boundaries are defined according to the IMGT scheme. Because framework (FR) regions constitute all of the variable domain sequence outside of the CDRs, once CDR boundaries are identified, framework regions are necessarily identified. The convention within the art is to label the framework regions as FR1 (sequence before CDR1), FR2 (sequence between CDR1 and CDR2), FR3 (sequence between CDR2 and CDR3), and FR4 (sequence after CDR3).

CDR and framework regions can also be demarcated using other numbering schemes and CDR definitions. The ABnum tool numbers the amino acid sequences of variable domains according to a large and regularly updated database called Abysis, which takes into account insertions of variable lengths and integrates sequences from Kabat, IMGT, and the PDB databases. The Honneger scheme is based on structural alignments of the 3D structures of immunoglobulin variable regions and allows one to define structurally conserved Ca positions and deduction of appropriate framework regions and CDR lengths (Honegger and Plückthun, J. Mol. Biol., 2001, 309:657-70). Similarly, Ofran et al. used a multiple structural alignment approach to identify the antigen binding residues of the variable regions called “Antigen Binding Regions (ABRs)” (Ofran et al., J. Immunol., 2008, 181:6230-5). ABRs can be identified using the Paratome online tool that identifies ABR by comparing the antibody sequence with a set of antibody-antigen structural complexes (Kunik et al. Nucleic Acids Res., 2012, 40:521-4). Another alternative tool is the proABC software, which estimates the probabilities for each residue to form an interaction with the antigen (Olimpieri et al., Bioinformatics, 2013, 29:2285-91).

In some embodiments, the CDRs of the antibodies of the invention are defined by the scheme or tool that provides the broadest or longest CDR sequence. In some embodiments, the CDRs are defined by a combination of schemes or tools that provides the broadest/longest CDRs. For example, from the Table 1 of CDR Definitions above, CDRL1 would be L24-L36, CDRL2 would be L46-L56, CDR3 would be L89-L97, CDRH1 would be H26-H35/H35B, CDR142 would be H47-H65, and CDR13 would be H93-H102. In some embodiments, the CDRs are defined by the Anticalign software, which automatically identifies all hypervariable and framework regions in experimentally elucidated antibody sequences from an algorithm based on rules from the Kabat and Chothia conventions (Jarasch et al., Proteins Struct. Funct. Bioinforma, 2017, 85:65-71). In some embodiments, the CDRs are defined by a combination of the Kabat, IMGT, and Chothia CDR definitions. In some embodiments, the CDRs are defined by the Martin scheme in combination with the Kabat and IMGT schemes. In some embodiments, the CDRs are defined by a combination of the Martin and Honneger schemes. In some embodiments, the CDRs comprise the ABR residues identified by the Paratome tool

Nucleic Acid Sequences

In some embodiments the antibodies or antigen-binding fragments thereof are administered as nucleic acids, including but not limited to mRNAs which could be modified and/or unmodified. See U.S. Pub 20180028645A1, U.S. Pub 20090286852, U.S. Pub 20130111615, U.S. Pub 20130197068, U.S. Pub 20130261172, U.S. Pub 20150038558, U.S. Pub 20160032316, U.S. Pub 20170043037, U.S. Pub 20170327842, U.S. Pat. Nos. 10,006,007, 9,371,511, 9,012,219, U.S. Pub 20180265848. U.S. Pub 20170327842, U.S. Pub 20180344838A1 at least at paragraphs [0260]-[0281], WO/2017/182524 for non-limiting embodiments of chemical modifications, wherein each content is incorporated by reference in its entirety.

mRNAs delivered in LNP formulations have advantages over non-LNPs formulations. See U.S. Pub 20180028645A1, WO/2018/081638, WO/2016/176330, wherein each content is incorporated by reference in its entirety. In certain embodiments the nucleic acid encoding an antibody or antigen-binding fragment thereof is operably linked to a promoter inserted an expression vector. In certain aspects the compositions comprise a suitable carrier. In certain aspects the compositions comprise a suitable adjuvant.

In certain embodiments the nucleic acid encoding an antibody or antigen-binding fragment thereof is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

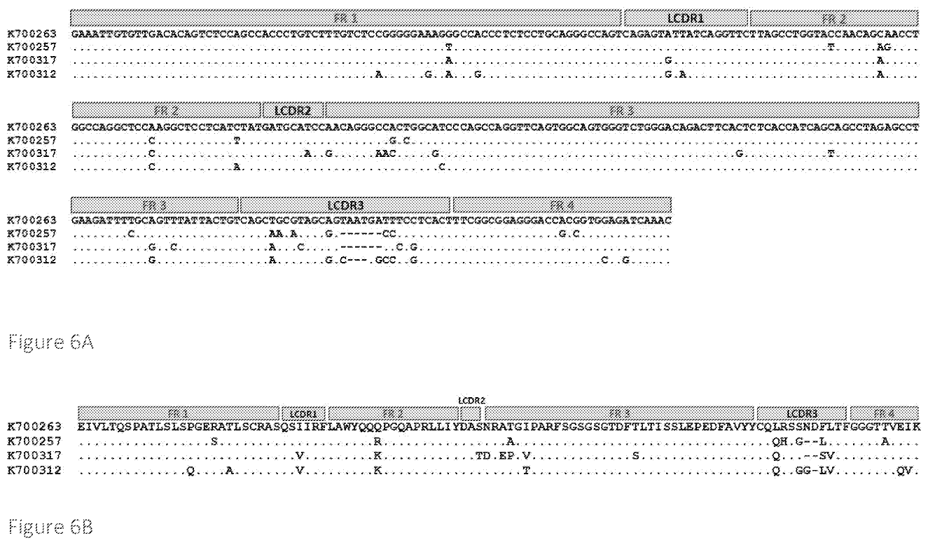

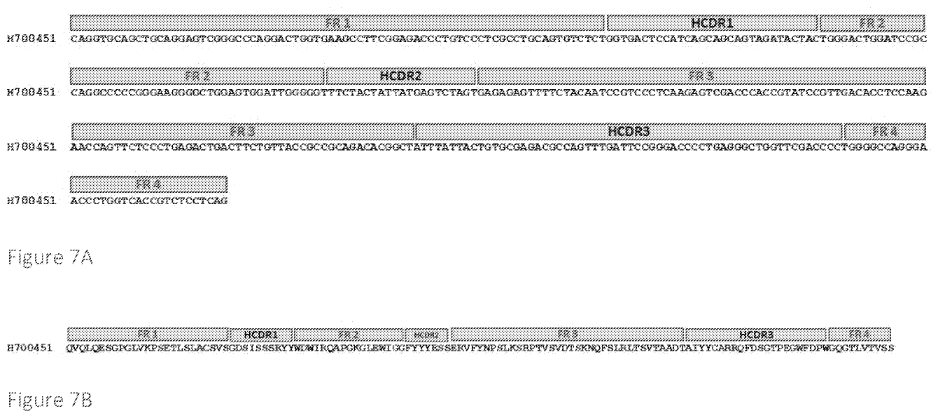

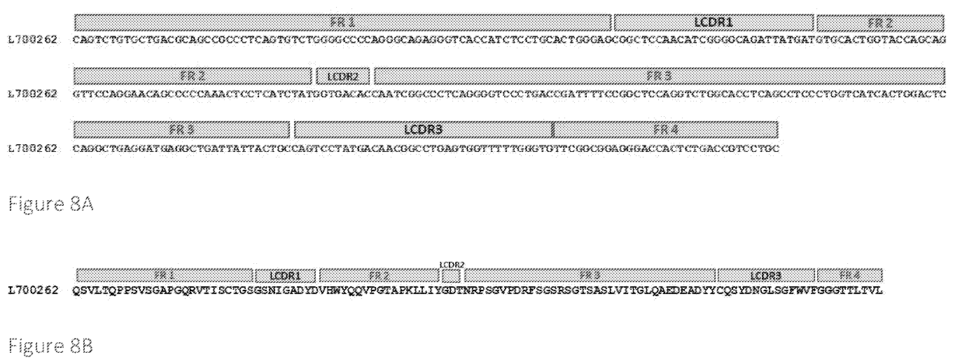

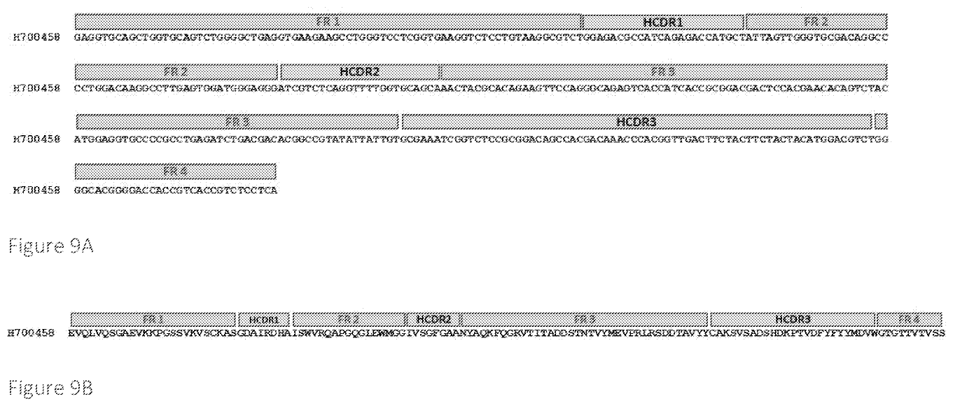

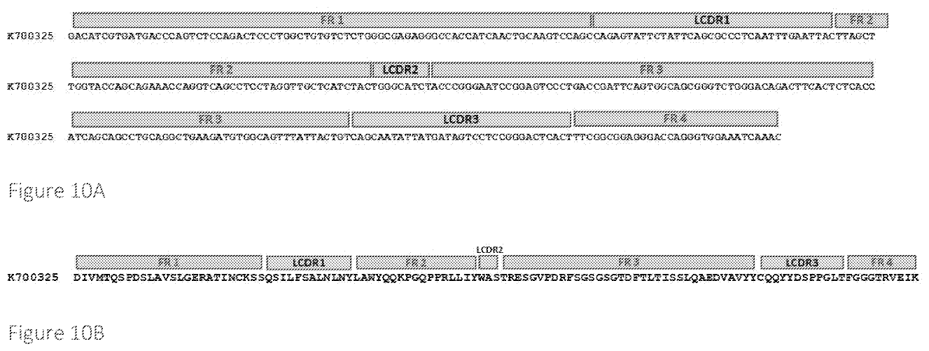

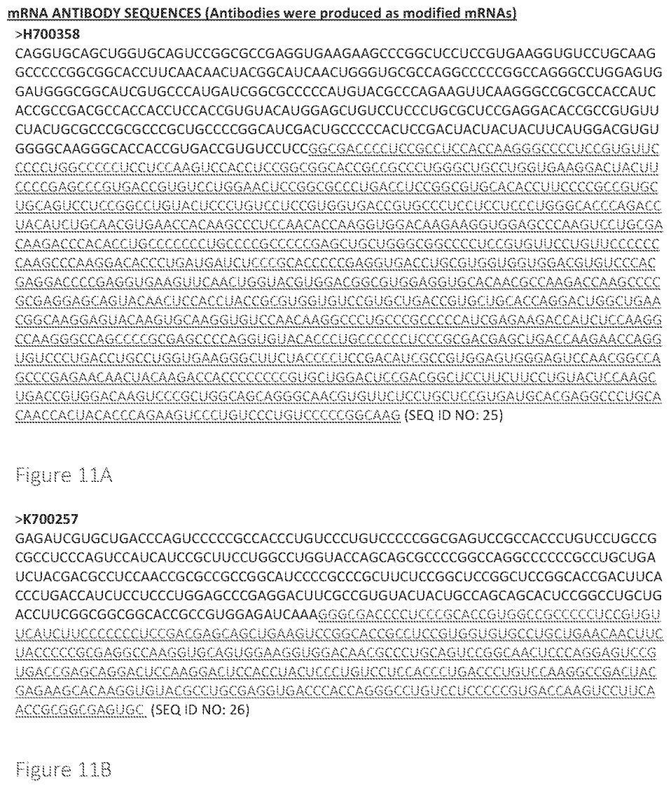

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding the polypeptide sequence of the sequences in FIG. 5-10, or a variant thereof or a fragment thereof. Accordingly, in one embodiment, the invention provides an RNA molecule encoding one or more of inventive antibodies or antigen-binding fragments thereof. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7 mG (5′)ppp(5′)NlmpNp). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. An RNA molecule may have a 3 poly-A tail. It may also include a poly-A polymerase recognition sequence (e.g. AAUAAA) near its 3′ end. In some embodiments, an RNA molecule useful with the invention may be single-stranded. In some embodiments, an RNA molecule useful with the invention may comprise synthetic RNA.

The recombinant nucleic acid sequence can be an optimized nucleic acid sequence. Such optimization can increase or alter the immunogenicity of the antibody or antigen-binding fragment thereof. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

Throughout the application, polynucleotides encoding an antibody or antigen-binding fragment thereof illustrate non-limiting embodiments of such nucleic acid sequences.

Animal Models

The mouse adapted virus challenge strain A/Aichi/2/1968 X-31 is known in the literature as a virus that contains the HA and NA of A/Hong Kong/1/1968 (H3N2) on the PR8 background.

Any other suitable flu animal model could be used to characterize the antibodies or antigen-binding fragments thereof of the invention. In non-limiting embodiments, a ferret flu animal model is used to characterize the antibodies.

Methods for pharmacokinetic evaluation of an antibody or antigen-binding fragment thereof in vivo are well known in the art.

Methods for in vitro transfection of mRNA and detection of antibody expression are known in the art.

CDRs as identified in the instant figures were identified using IMGT.

### EXAMPLES

### Example 1: H3N2 Neutralizing Antibodies

Antibody Isolation: The host immune response to vaccination and/or natural exposure to viral infection is often evaluated using surrogate viral antigens such as peptides, recombinant (r) proteins, pseudo-viruses, virus like particles (VLPs), etc. While powerful, there are several significant drawbacks associated with these strategies including: 1) need for pre-existing rDNA expression vectors, 2) temporal lag in reagent availability associated with synthesizing/generating new recombinant platforms, 3) antigenic dissimilarities associated with conformational differences between recombinantly expressed proteins and viral particles, and 4) lack of sensitivity in detecting antibodies that bind/neutralize via interactions with complex native viral epitopes.

The whole virus particle in its native conformation is the ideal “antigen” for detailed characterization of a virus-specific functional humoral immune response. The detection of virus particles and/or virus-infected cells can prove challenging as it often requires the presence of preexisting virus-specific immunodetection reagents. Here we used a method for efficient labeling, semi-purification, and concentration of enveloped viruses using fluorescent lipid dyes.

The commercial availability of fluorescent (fluor-) lipid dyes of various excitation/emission spectra as well as biotinylated versions creates a flexible methodology that can be tailored to integrate with existing downstream methodologies using additional fluor-conjugated detection reagents (fluorescence activated cell sorting (FACS), fluorescence microcopy, etc.). Fluorescent-lipid-labeled whole virus particles can be utilized to identify potently neutralizing virus-specific antibodies, assess purified antibody/virus interactions (binding and neutralization assays), and expedite assay turnaround times. This methodology can generate semi-purified and concentrated fluorescent-lipid-labeled whole virus particles from crude culture preparations of any enveloped virus using commercially available reagents and standard equipment available in any virology laboratory. A commercially available reagent—Vybrant™ Cell Labeling Solution (DiO, Dil, DiD, Invitrogen #V22889)—was used to stain whole flu virus used for the cell sorting experiments.

Human PBMC were used for antibody isolation from a donor vaccinated with the 2017 seasonal influenza vaccine. PBMCs were collected seven days after immunization and cryopreserved. Cryopreserved PBMCs were thawed, stained with a fluorescently conjugated antibody panel to identify B cells and fluorescently labeled whole A/Hong Kong/4801/2014 virus to detect B cells with flu-specific surface immunoglobulin (pathogen-specific B Cell Receptors; BCR). Virus labeling process was developed with commercial reagents. Virus-specific B cells were single-cell sorted into PCR plates (FIG. 1). The BCR variable heavy and light chain genes were reverse transcription PCR-amplified as previously described¹ but with the following modifications: reverse transcription was performed using random hexamers, first round heavy chain primer and protocol was provided by the NIH-VRC and the nested PCR reactions (2^{nd} PCR/PCRb) was performed by multiplexing. Ig linear expression cassettes (Ref 1) were generated for 135 candidates.

Antibody Binding and neutralization: These antibodies were transiently transfected and assayed in ELISA for binding to a panel of 14 strain-specific recombinant HA proteins, 1 matrix protein and 2 neuraminidase proteins. The A/Hong Kong/4801/2014 HA protein was used because it matched the virus used for flow cytometry sorting. The other HA proteins used were those commercially available.

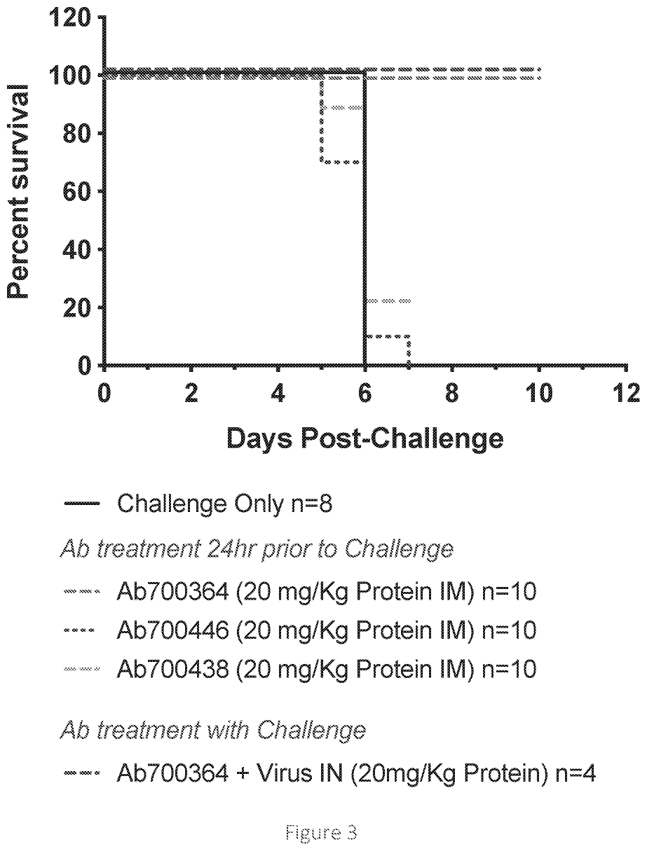

A total of 25 out of 135 candidates bound HA proteins and no antibodies bound to either matrix or neuraminidase proteins. The 25 HA-reactive antibodies were tested for neutralization in assays with A/Hong Kong/4801/2014—the virus used for B cell sorting. Six antibodies—Ab700364, Ab700358, Ab700446, Ab700438, Ab700451, Ab700458—were identified with potent A/Hong Kong/4801/2014 neutralization. A DNA plasmid order was placed with Genscript and all six antibodies were transiently transfected in larger scale and antibody was Protein A purified for repeat neutralization assays. In addition, the plasmids were also used by the Duke Human Vaccine Institute Protein Production Facility to produce larger quantities of antibody. All six antibodies were then assessed for binding to 14H3, 5 H1, 2 H5, 1 H7 and 1H18 strain-specific HA proteins screened. Of the 14H3, 5 H1, 2 H5, 1 H7 and 1H18 strain-specific HA proteins screened, the antibodies only bound to H3 HA protein (FIG. 2). Ab700358 bound 9 of 14, Ab700458 bound 7 of 14, Ab700438 bound 8 of 14, Ab700446 bound 8 of 14, Ab700364 bound 8 of 14 and Ab700451 bound 3 of 14H3 proteins. As expected, all antibodies bound to the H3 HA derived from the Influenza A/Hong Kong/7/4801/2014 strain.

In vitro microneutralization assays (Refs 2-4) were used to determine the functional efficacy of these antibodies to neutralize influenza viruses. All six antibodies potently neutralized A/Hong Kong/7/4801/2014 virus (minimum effective concentration (MEC) 0.004-0.007 μg/mL) and more weakly neutralized the mouse adapted H3N2 influenza strain A/Aichi/2/1968 X-31 (minimum effective concentration >50-0.78 μg/mL).

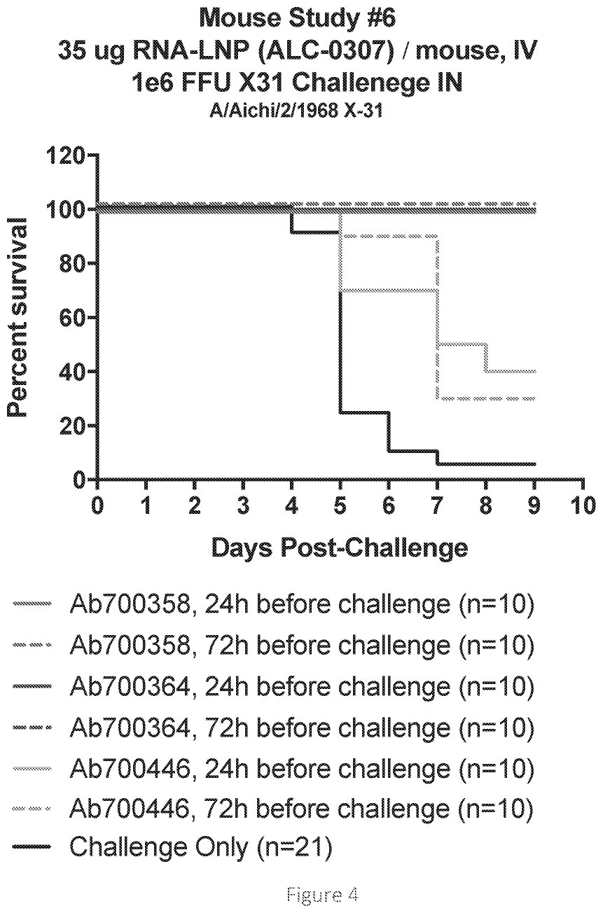

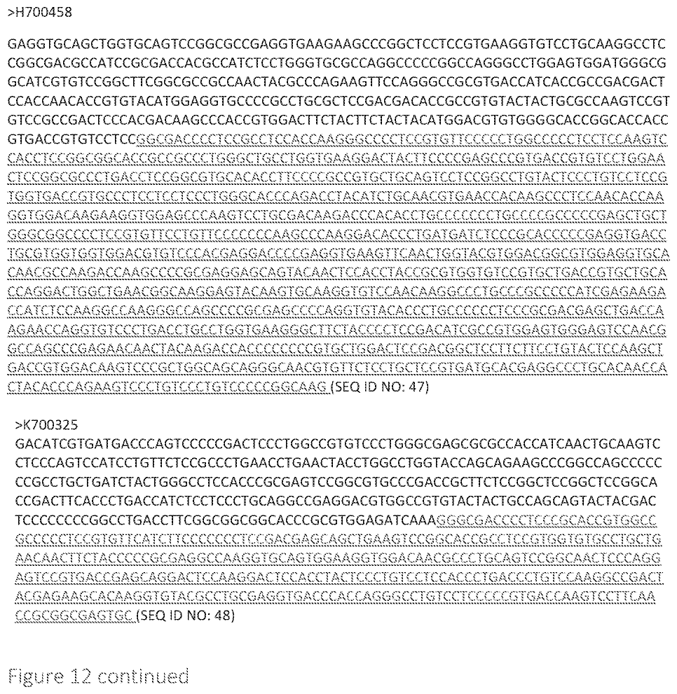

In vivo influenza virus challenge study with recombinant protein antibodies: A/Aichi/2/1968 X-31 is a mouse adapted influenza, virus strain. Recombinantly expressed antibodies Ab700364, Ab700358, Ab700446, Ab700438 and Ab700458 neutralized this viral strain in vitro with an MEC 0.78-25 μg/mL (FIG. 13) and Ab700364, Ab70046 and Ab700438 were used for an in vivo mouse virus challenge study. Ten mice per group were treated intramuscularly with 20 mg/kg of antibody protein 24 hours prior to virus challenge. Animals were challenged by intranasal administration of 1×10⁶ FFU of A/Aichi/2/1968 X-31. In addition, four mice received 20 mg/kg of Ab700364 pre-mixed with 1×10⁶ FFU of A/Aichi/2/1968 X-31 and delivered intranasally. The mice that received no antibody or received the weakly neutralizing antibodies Ab700446 or Ab700438 did not survive past 7 days. Conversely the mice that received the best neutralizing antibody Ab700364 were 100% protected by either pre-administration of antibody or intranasal co-delivery (FIG. 3).

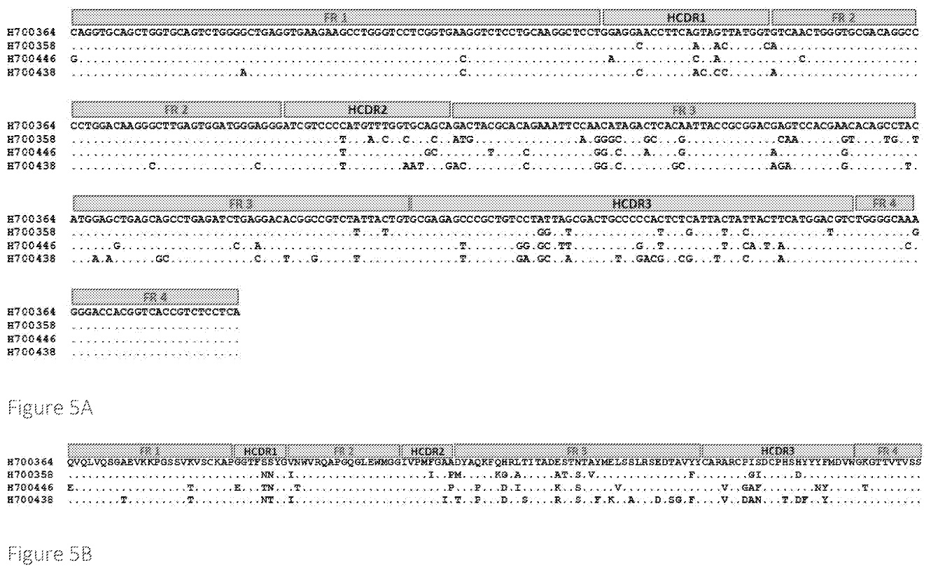

In vivo influenza virus challenge study with recombinant mRNA encoded antibodies: Mice were treated IV with 35 μgs of modified mRNA encoded Ab700358, Ab700364 or Ab700446 formulated in lipid nanoparticles or no mRNA-LNP. Either 24 hours or 72 hours after mRNA-LNP delivery mice were challenged by intranasal administration of 1×10⁶ FFU of A/Aichi/2/1968 X-31. Mice that received Ab700358 and Ab700364 were completed protected from either 24 or 72 hour challenge. Animals that received Ab700446 were partially protected (FIG. 4).

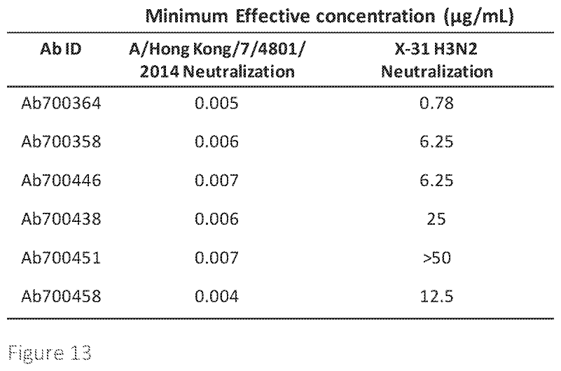

Antibody Sequences: Sanger sequenced antibodies were analyzed with Cloanalyst (Ref 5). Based on this analysis, antibodies Ab700364, Ab700358, Ab700446 and Ab700438 belong to the same clonal lineage. Ab700451 and Ab700458 are single member clones. Ab700364 was the least mutated member of the four-member clonal lineage, the most potent A/Aichi/2/1968 X-31 neutralizing antibody and the only antibody that protected in vivo at the dose tested. The immunogenetics of the antibodies are shown in FIG. 14.

In Vivo Pharmacokinetics of mRNA/LNP Flu Countermeasure in Non-Human Primates

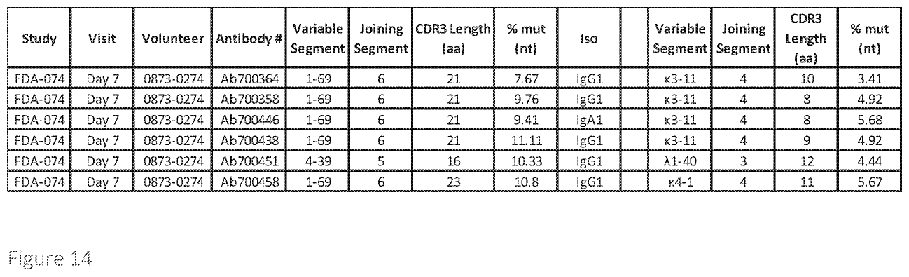

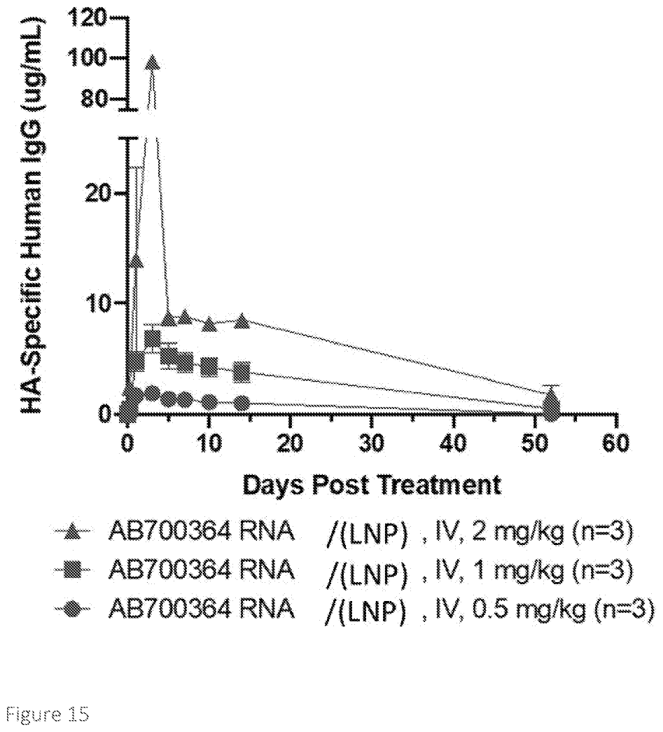

FIG. 15 shows Modified mRNA expressing AB700364 (anti-H3N2) was formulated in lipid nanoparticle (LNP) and administered to NHPs at three doses (2, 1, 0.5 mg RNA/kg) via the intravenous (IV) route to determine human antibody expression in vivo when launched off mRNA. Serum antibody levels were determined at 6 h, 1 d, 3 d, 7 d, 10 d, 14 d and 52 d after IV administration. Using a luminex bead-based assay, A/Hong Kong/4801/2014 H3N2 HA specific human IgG was quantified in serum samples post treatment.

Antibody levels peaked at around 1 day post administration, was maintained up to 14 day post administration and returned to baseline at 52 days (FIG. 15). These human antibody expression kinetics from a single dose of mRNA mRNA are consistent with our mouse studies and prior art with respect to human IgG1 half-life in Non-human primates.

### REFERENCES

1. Liao H X, Levesque M C, Nagel A, et al. High-throughput isolation of immunoglobulin genes from single human B cells and expression as monoclonal antibodies. J Virol Methods. 2009; 158 (1-2):171-179.
2. Bajic G. Maron M J, Adachi Y, et al. Influenza Antigen Engineering Focuses Immune Responses to a Subdominant but Broadly Protective Viral Epitope. Cell Host Microbe. 2019; 25(6):827-835 e826.
3. Watanabe A, McCarthy K R, Kuraoka M, et al. Antibodies to a Conserved Influenza Head Interface Epitope Protect by an IgG Subtype-Dependent Mechanism. Cell. 2019; 177(5):1124-1135 e1116.
4. McCarthy K R, Watanabe A, Kuraoka M, et al. Memory B Cells that Cross-React with Group 1 and Group 2 Influenza A Viruses Are Abundant in Adult Human Repertoires. Immunity. 2018; 48(1):174-184 e179.
5. Kepler T B, Munshaw S, Wiehe K, et al. Reconstructing a B-Cell Clonal Lineage. II. Mutation, Selection, and Affinity Maturation. Front Immunol. 2014; 5:170.

Ab700364 clonal lineage consists of Ab700364, Ab700358, Ab700446 and Ab700438 which are in the same lineage. The antibodies in the Ab7000364 clonal lineage are mutated (7%-11%).

Table 3 below shows the names of each Vh and Vl comprising the respective antibodies.

| Ab ID | IgH_ID | IgL_ID |
|---|---|---|
| Ab700364 | H700364 | K700263 |
| Ab700358 | H700358 | K700257 |
| Ab700446 | H700446 | H700317 |
| Ab700438 | H700438 | K700312 |
| Ab700451 | H700451 | L700262 |
| Ab700458 | H700458 | K700325 |

Table 4 shows correlation of antibodies sequences and FIGS.

| | IgH | IgH | IgL | IgL |
|---|---|---|---|---|
| | sequence | sequence | sequence | sequence |
| Ab | DNA FIG. | RNA FIG. | DNA FIG. | RNA FIG. |
| Ab700364 | 12 | 11C | 12 | 11D |
| Ab700358 | 12 | 11A | 12 | 11B |
| Ab700446 | 12 | 11G | 12 | 11H |
| Ab700438 | 12 | 11E | 12 | 11F |
| Ab700451 | 12 | 11I | 12 | 11J |
| Ab700458 | 12 | 11K | 12 | 11L |

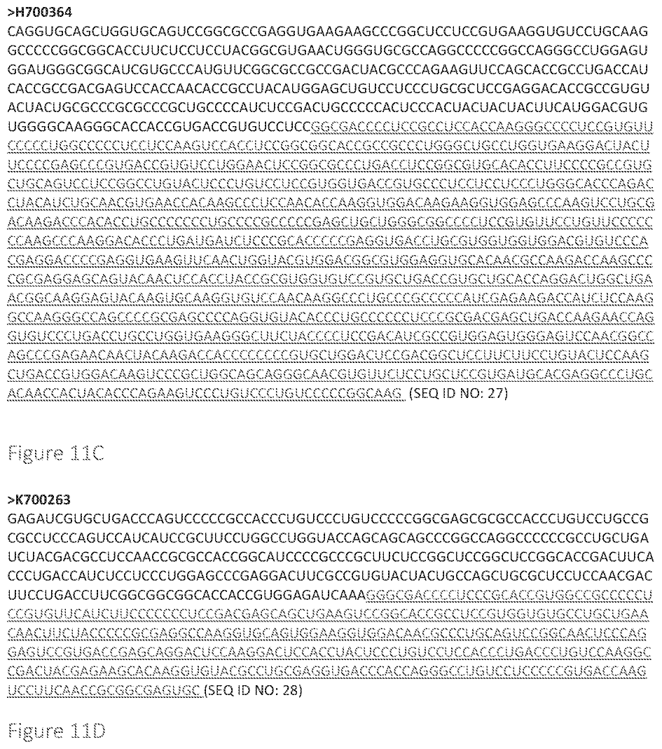

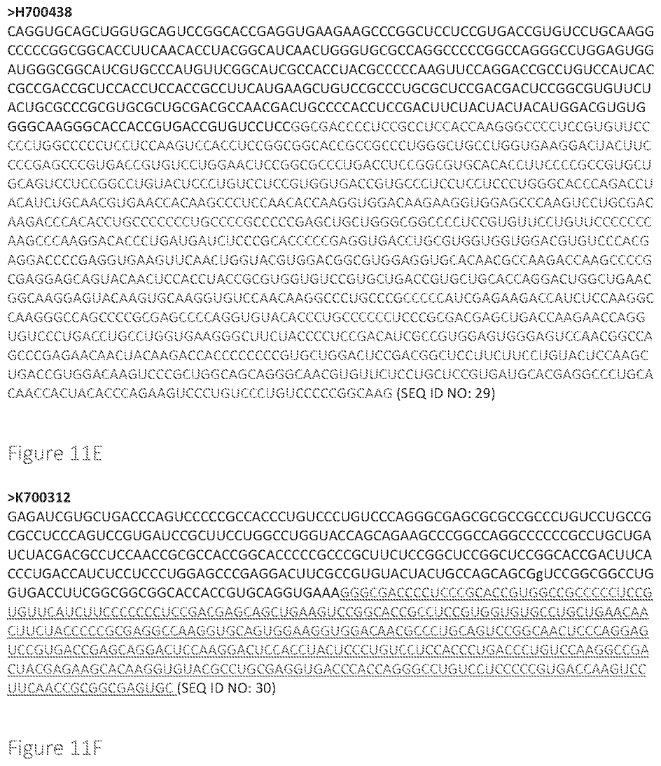

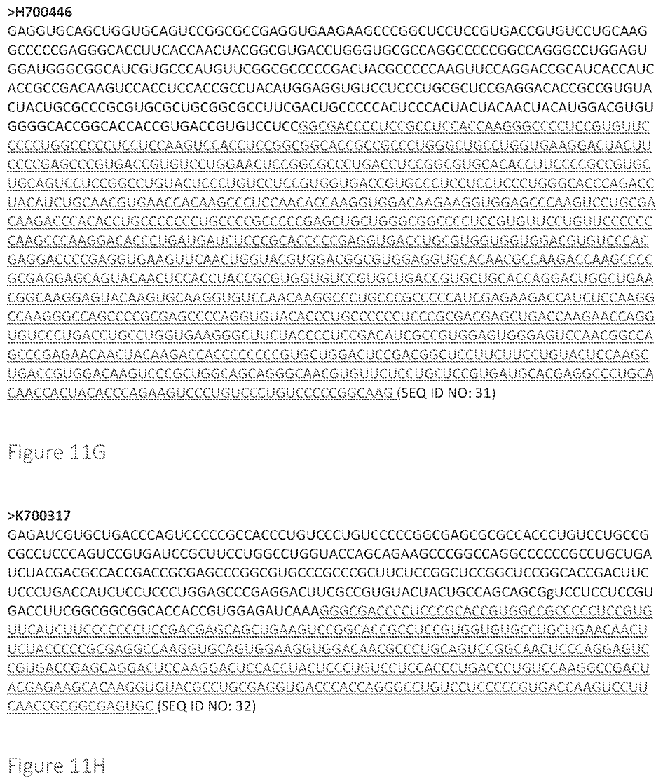

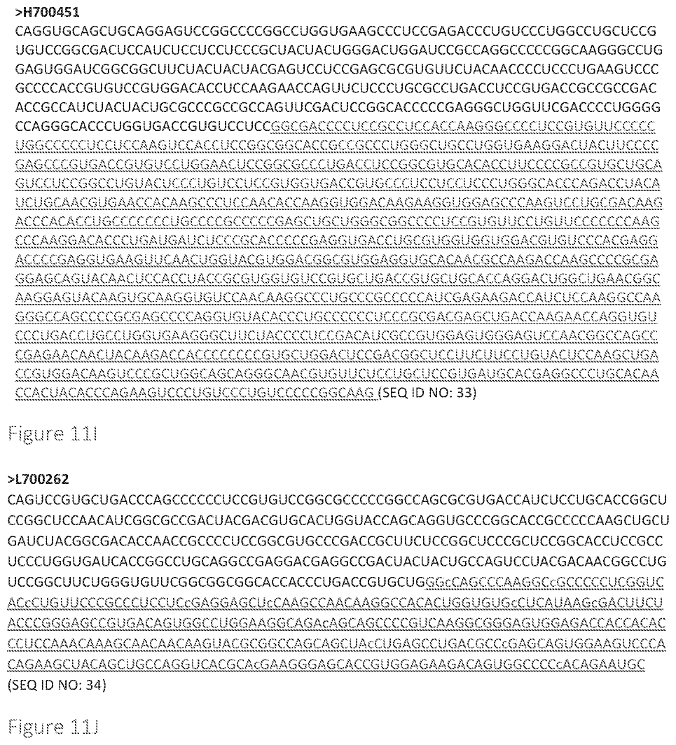

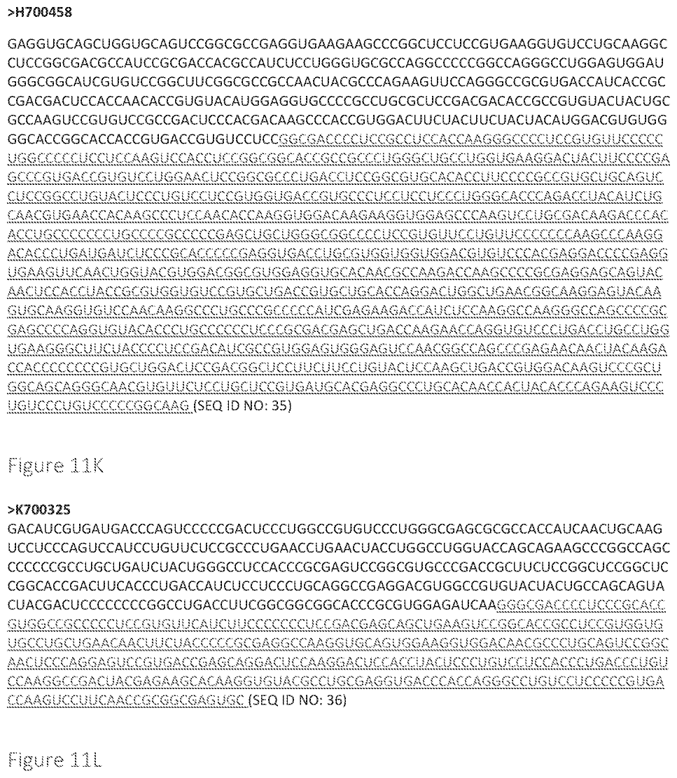

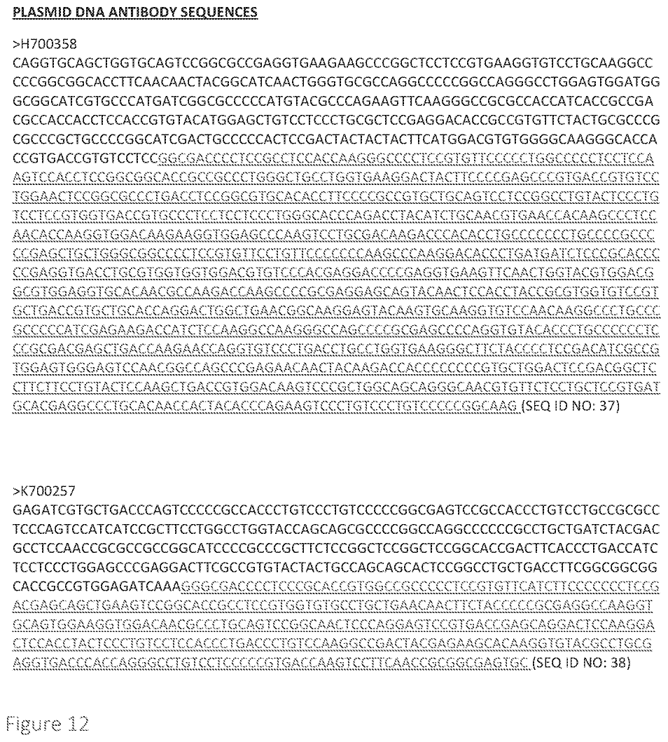

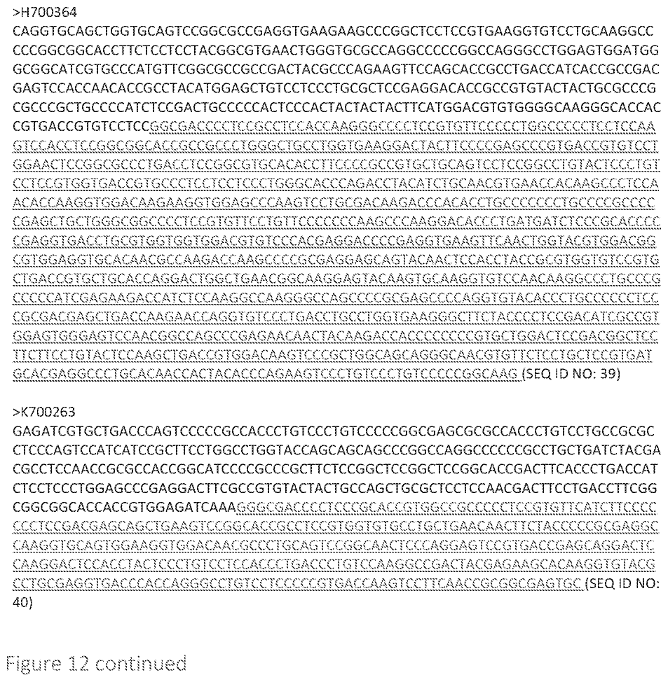

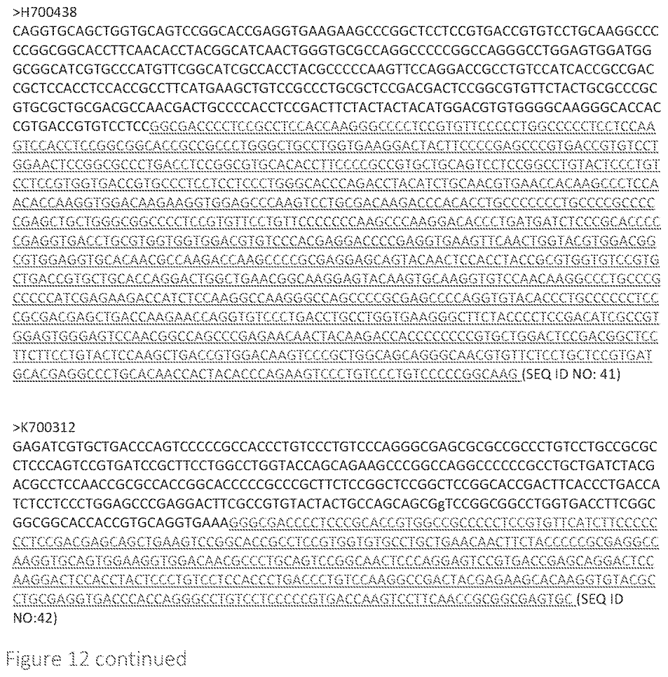

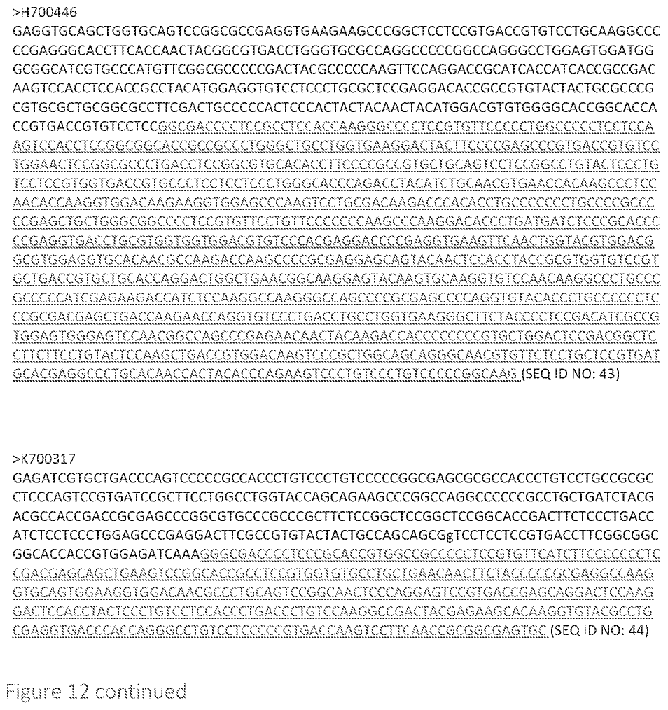

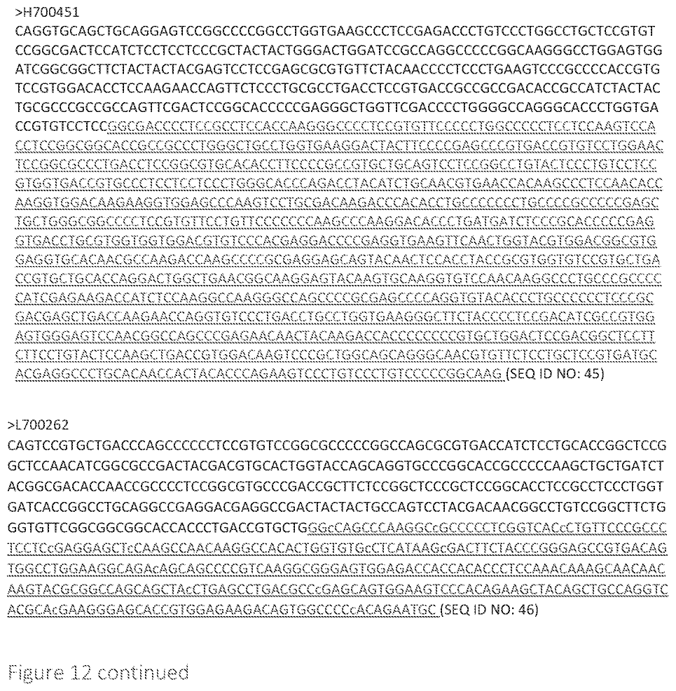

FIGS. 11 and 12 show non-limiting embodiments of nucleic acids encoding antibodies of the invention. In FIGS. 11A-L-12: Black nucleotides correspond to the variable region. In FIGS. 11A, 11C, 11E, 11G, 11I, 11K, FIG. 12 SEQ ID NOs: 37, 39, 41, 43, 45, and 47, blue nucleotides are underlined and correspond to the IgG1 LS constant region. For LS mutation See e.g. Gaudinski M R. Coates E E, Houser K V, Chen G L, Yamshchikov G, Saunders J G, et al. (2018) Safety and pharmacokinetics of the Fe-modified HIV-1 human monoclonal antibody VRC01LS: A Phase 1 open-label clinical trial in healthy adults. PLoS Med 15(1): e1002493 (doi.org/10.1371/journal.pmed.100249). In FIGS. 11B, 11D, 11F, 11H, 11L, FIG. 12 SEQ ID NOs: 38, 40, 42, 44, 48, green nucleotides are underlined and correspond to the IgK constant region. In FIG. 11J and FIG. 12 SEQ ID NO: 46 purple nucleotides are underlined and correspond to the IgL constant region.

In one embodiment, the IgG constant region comprises the LS mutation. Additional variants of the Fc portion of the antibody are also contemplated by the invention. See Maeda et al. MAbs. 2017 July; 9(5): 844-853. Published online Apr. 7, 2017, PMID: 28387635.

What is claimed is:

1. A recombinant influenza antibody, or an antigen-binding fragment thereof, wherein in certain non-limiting embodiments the antibody or antigen-binding fragment thereof specifically binds to an influenza virus and neutralizes influenza virus infection.

2. The recombinant influenza antibody, or the antigen-binding fragment thereof, according to claim 1, wherein the antibody, or antigen-binding fragment thereof binds to H3N2 virus.

3. The recombinant influenza antibody, or the antigen-binding fragment thereof, according to any of claims 1-2, wherein the antibody, or antigen-binding fragment thereof are H3 HA specific.

4. The recombinant influenza antibody, or the antigen-binding fragment thereof, according to any of claims 1-3, wherein the antibody or antigen-binding fragment thereof, is a fully human antibody.

5. The recombinant influenza antibody, or the antigen-binding fragment thereof, according to any of claims 1-4, wherein the antibody, or antigen-binding fragment thereof, is a recombinant human monoclonal antibody.

6. The recombinant influenza antibody, or the antigen-binding fragment thereof, according to any of claims 1-5, wherein the antibody, or antigen-binding fragment thereof, comprises an Fc moiety.

7. The recombinant influenza antibody, or the antigen-binding fragment thereof, according to claim 6, wherein the antibody, or antigen-binding fragment thereof, comprises a mutation(s) in the Fc moiety, optionally the mutation reducing binding of the antibody or antigen-binding fragment thereof to an Fc receptor, optionally the mutation increasing the half-life of the recombinant antibody or antigen-binding fragment thereof.

8. A recombinant influenza antibody or antigen-binding fragment thereof, as described in Example 1, FIG. 14, or FIGS. 5-10.

9. The recombinant influenza antibody, or antigen-binding fragment thereof, according to any of claims 1-8, wherein the antibody or antigen-binding fragment thereof, comprises a heavy chain (VH) comprising CDRH1, CDRH12 and CDRH3 and a light chain (VL) comprising CDRL1, CDRL2 and CDRL3, wherein each CDR comprises an amino acid sequence according to any of the sequences listed in FIG. 5-10, or a functional sequence variant thereof having at least 70%, at least 75%, at least 80%, at least 85%, at least 88%, at least 90%, at least 92%, at least 95%, at least 96%, at least 97%, at least 98% or at least 99% sequence identity.

10. The recombinant influenza antibody, or antigen-binding fragment thereof according to claim 9, wherein the antibody or antigen-binding fragment thereof, comprises a heavy chain comprising CDRH1, CDRH2 and CDRH13 and a light chain comprising CDRL1, CDRL2 and CDRL3, according to any of the sequences of VH H700364 or of VL K7000263 in FIGS. 5A and 5B, or a functional sequence variant thereof having at least 70%, at least 75%, at least 80%, at least 85%, at least 88%, at least 90%, at least 92%, at least 95%, at least 96%, at least 97%, at least 98% or at least 99% sequence identity.

11. The recombinant influenza antibody, or antigen-binding fragment thereof, according to claim 10, wherein the antibody or antigen-binding fragment thereof, comprises a VH amino acid sequence VH H700364 and a VL amino acid sequence VL K7000263 in FIGS. 5A and 5B or a functional sequence variant thereof having at least 70%, at least 75%, at least 80%, at least 85%, at least 88%, at least 90%, at least 92%, at least 95%, at least 96%, at least 97%, at least 98% or at least 99% sequence identity.

12. The recombinant influenza antibody, or antigen-binding fragment thereof according to claim 11, wherein the antibody or antigen-binding fragment thereof, comprises a VH amino acid sequence according to VH H700364 and a VL amino acid sequence according to VL K7000263 in FIGS. 5A and 5B or a functional sequence variant thereof having at least 95%, at least 96%, at least 97%, at least 98%, or at least 99% sequence identity.

13. The recombinant influenza antibody, or antigen-binding fragment thereof according any of the preceding claims, wherein the antibody or antigen-binding fragment thereof, comprises a VH amino acid sequence according to VH H700364 and a VL amino acid sequence according to VL K7000263 in FIGS. 5A and 5B.

14. The recombinant influenza antibody, or antigen-binding fragment thereof, according to any of the previous claims, wherein the antibody, or the antigen-binding fragment thereof, is a purified antibody, a single chain antibody, Fab, Fab′, F(ab′)2, Fv or scFv.

15. The recombinant influenza antibody or antigen-binding fragment thereof of any of the preceding claims wherein the antibody or antigen-binding fragment thereof is any isotype.

16. The recombinant influenza antibody, or antigen-binding fragment thereof, according to any of the previous claims, for use as a medicament.

17. The recombinant influenza antibody, or antigen-binding fragment thereof, according to any of the previous claims, for use in the prevention and/or treatment of influenza virus infection.

18. A nucleic acid molecule comprising a polynucleotide encoding the influenza antibody, or the antigen-binding fragment thereof, according to any of the previous claims.

19. The nucleic acid molecule according to claim 18, wherein the polynucleotide sequence comprises, consists essentially of, or consists of a nucleic acid sequence according to any one of the sequences in FIG. 5-10; or a functional sequence variant thereof having at least 70%, at least 75%, at least 80%, at least 85%, at least 88%, at least 90%, at least 92%, at least 95%, at least 96%, at least 97%, at least 98% or at least 99% sequence identity.

20. The nucleic acid of claim 18 or 19, wherein the nucleic acid a ribonucleic acids (RNA) as shown in FIGS. 11A-11L.

21. A vector comprising the nucleic acid molecule according to claims 18-20.

22. A cell expressing the influenza antibody, or antigen-binding fragment thereof, according to any of the preceding claims; or comprising the vector according to claim 21.

23. A pharmaceutical composition comprising the recombinant influenza antibody, or antigen-binding fragment thereof, according to any of the preceding claims, the nucleic acid according to claims 18-20, the vector according to claim 21 and/or the cell according to claim 22, and optionally further comprising a pharmaceutically acceptable carrier.

24. The pharmaceutical composition according to claim 23, further comprising a pharmaceutically acceptable excipient, diluent, or carrier.

25. A method of treating or preventing influenza infection in a subject in need thereof, comprising administering the recombinant influenza antibody or antigen-binding fragment thereof of any of the preceding claims, the nucleic acid of any one of claim 18-20, the vector of claim 21, or the pharmaceutical composition of claim 23-24 in an amount suitable to effect treatment or prevention of influenza infection.

26. The method of claim 25, wherein the recombinant influenza antibody or antigen-binding fragment thereof is administered prior to influenza exposure or at the same time as influenza exposure.

27. An in vitro transcription system to synthesize ribonucleic acids (RNAs) encoding influenza antibodies or antigen-binding fragments of the invention, comprising: a reaction vessel, a DNA vector template comprising a nucleic acid sequence encoding an influenza antibody or antigen-binding fragment of the invention as described in any of the preceding claims, and reagents for carrying out an in vitro transcription reaction that produces mRNA encoding an influenza antibody or antigen-binding fragment thereof of the invention, wherein optionally the mRNA is modified mRNA.

28. A method for manufacturing an mRNA encoding an influenza antibody or antigen-binding fragment thereof, comprising:
a. providing an in vitro transcription reaction vessel comprising a DNA template encoding an influenza antibody or antigen-binding fragment thereof according to any of the preceding claims and reagents under conditions suitable for in vitro transcription of the nucleic acid template, thereby producing an mRNA template encoding the influenza antibody or antigen-binding fragment thereof according to any of the preceding claims, and
b. isolating the mRNA by any suitable method of purification and separating reaction reagents, the DNA template, and/or mRNA product related impurities.

29. A method for manufacturing an influenza antibody or antigen-binding fragment thereof, comprising culturing a host cell comprising a nucleic acid according to any of the preceding claims under conditions suitable for expression of the influenza antibody or antigen-binding fragment thereof and isolating said influenza antibody or antigen-binding fragment thereof.
